SEQUENCE LISTING (3 sequences)

SEQ ID NO: 1 - protein, 7 aa, Artificial Sequence
  chemically synthesized
  misc_feature (4)..(4): Xaa can be any naturally occurring amino acid
  misc_feature (6)..(6): Xaa can be any naturally occurring amino acid
YSYXTXV

SEQ ID NO: 2 - protein, 7 aa, Artificial Sequence
  chemically synthesized
YSYFTVV

SEQ ID NO: 3 - protein, 7 aa, Artificial Sequence
  chemically synthesized
YSYYTIV

(19) United States
(12) Patent Application Publication
(10) Pub. No.: US 2020/0306337 A1
(43) Pub. Date: Oct. 1, 2020

(54) Bioactive Metabolities Encoded by the Human Microbiome Using Primary Sequence Alone

(71) Applicant: The Rockefeller University, New York, NY (US)

(72) Inventor: Sean F. Brady, New York, NY (US)

(21) Appl. No.: 16/300,127

(22) PCT Filed: May 9, 2017
(86) PCT No.: PCT/US2017/031677

§ 371 Date: Nov. 9, 2018

(51) Int. Cl. A61K 38/08; A61P 31/04

(52) U.S. Cl. CPC A61K 38/08; A61P 31/04

(57) ABSTRACT

The present invention relates to a bioinformatic method of predicting a desired compound based on genetic information and the chemical synthesis of the predicted compound.

### CROSS-REFERENCE TO RELATED APPLICATIONS

This application claims priority to and the benefit of U.S. Application No. 62/333,497, filed May 9, 2016, the content of which is incorporated in its entirety herein.

### BACKGROUND OF THE INVENTION

Many important secondary metabolites in bacteria are synthesized by modular biosynthetic gene-clusters: polyketide synthases (PKS), non-ribosomal peptide synthetases (NRPS), NRPS-independent siderophore synthetases (NIS) and hybrid synthase/synthetase gene-clusters. Other biologically active compounds including antibiotics comprise polypeptides assembled by NRPSs. The biosynthesis of these compounds occurs by a series of sequential steps and each step is encoded by a group of catalytic domains grouped as different modules. NRPSs typically show a modular architecture and tethered biosynthetic strategy (Cane et al, 1998; Keating and Walsh, 1999). The modules are usually arranged across several polypeptides, with often more than one module in a polypeptide. NRPS modules assemble amino acids (Finking and Marahiel, Ann. Rev. Microbiol., 58, 453-488, 2004; Challis, ChemBioChem, 6, 601-611, 2005). The modules are composed of catalytic domains with standard (condensation, adenylation and peptidyl carrier protein; C-A-PCP) domains present in all modules as well as optional domains (e.g. epimerization (Epi), cylization (Cyc), N-methylation (N-Meth), domains etc.). In NRPSs a condensation (C) domain removes the growing peptide unit from the upstream PCP domain and couples it to the next amino acid group in the chain, which has already been selected and loaded by an adenylation (A) domain onto the PCP in the same module (Marahiel et al, 1997; Stachelhaus et al, 1998). Other catalytic domains (e.g., epimerase or N-methytransferase) within an elongation module can modify the newly elongated polypeptide before it is transferred to the next module in the biochemical assembly line (Marahiel et al, 1997). In addition to the 20 amino acids present in most proteins, NRPSs may catalyze the formation of polypeptides composed of other amino acids.

The characterization of small molecules produced by bacteria in laboratory culture has been a key step to understanding bacterial physiology and developing small molecule therapeutics (Newman and Cragg, J. Nat. Prod. 2012, 75:311). As successful as this approach has been for identifying novel bioactive small molecules, extensive sequencing of bacterial genomes and metagenomes has revealed that the bacterial biosynthetic diversity currently accessed in the laboratory represents only a small fraction of what is predicted to exist in nature (Charlop-Powers et al., Curr. Opin. Microbiol. 2014, 19:70). This shortcoming arises from an inability to culture most bacteria in the laboratory and from the fact that most biosynthetic gene clusters remain silent under laboratory fermentation conditions.

Large scale DNA sequencing has revealed many modular gene-clusters, whose products are not known (Bentley et al, Nature, 417, 141-147, 2002; Ikeda et al, Nat. Biotechnol. 21, 526-531, 2003; Oliynyk et al, Nat. Biotechnol, 25, 447-453, 2007). The genome sequencing of bacteria is expanding rapidly with over 4000 genome sequences publicly available at present. Current advances in sequencing technology will lead to an explosive increase in known genome sequences in the near future. Moreover, recent developments in DNA sequencing technology have opened up new opportunities allowing the DNA sequencing of unculturable microorganisms. This field of study, called metagenomics, allows access to the genetic potential of culturable and unculturable microorganisms (see references in: Dunlap et al, Curr. Med. Chem., 13, 697-710, 2006). There are several hundred sequenced modular gene-clusters in public databases that have not been adequately analyzed and their number will grow exponentially with every new bacterial genome and metagenome sequenced. Traditional screening approaches will miss many interesting compounds, because they are not produced under the fermentation conditions used. Additionally, purification of an unknown compound is costly and time-intensive.

Thus there is a need in the art for a compound discovery pipeline that avoids the requirement for either bacterial culture or gene cluster expression. The present invention satisfies the need in the art.

### SUMMARY OF THE INVENTION

The invention provides a composition comprising a peptide, wherein the peptide comprises an amino acid sequence of YSY[X1]T[X2]V (SEQ ID NO: 1) or any derivative or analog thereof.

In one embodiment, the peptide comprises an N-terminal modification.

In one embodiment, the N-terminal modification comprises an acyl group on the N-terminus of the peptide.

In one embodiment, the N-terminal modification comprises a fatty acid on the N-terminus of the peptide.

In one embodiment, the fatty acid is β-hydroxymyristic acid (HMA).

In one embodiment, X₁ is F or Y.

In one embodiment, X₂ is V or I.

In one embodiment, the peptide comprises an amino acid sequence selected from the group consisting of YSYFTVV (SEQ ID NO: 2) and YSYYTIV (SEQ ID NO: 3).

In one embodiment, Y at position 1, S at position 2, X₁ at position 4, X₂ at position 6, and V at position 7 are in the L configuration.

In one embodiment, Y at position 3 and T at position 5 are in the D configuration.

In one embodiment, the composition is an antibiotic.

In one embodiment, the composition further comprises a pharmaceutical acceptable carrier.

In one embodiment, the peptide is synthetically synthesized.

The invention also provides a method of inhibiting proliferation of a bacterial cell, the method comprising administering to the bacterial cell any of the compositions of the invention.

In one embodiment, the bacterial cell is Gram-positive.

In one embodiment, the bacterial cell is in a mammal.

In one embodiment, the mammal is a human.

In one embodiment, the mammal is a non-human.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A to 1C, is a flow diagram depicting the Syn-BNP antibiotic discovery process. FIG. 1A depicts the process of prediction of compounds from primary sequence data alone through chemical synthesis of the predicted compounds. FIG. 1B depicts the application of the syn-BNP approach to NRPs predicted from human microbiome sequence data and assessment of new molecules for antibiosis activities. FIG. 1C depicts one exemplary mode of action for novel compounds of the invention, as serving to control the assemblage of microbes present in a consortia through the production of narrow spectrum antibiotics.

FIG. 2 is a table providing a list of syn-BNPs.

FIG. 3, comprising FIGS. 3A to 3E, depicts the development and testing of the humimycins as antibiotic peptides. FIG. 3A depicts closely related gene clusters found in two Rhodococcus spp. cultured from human subjects. FIG. 3B depicts the chemical structures of humimycin A (1) and B (2). The two antibiotic peptides differ at the fourth (F/Y) and sixth (V/I) residues. FIG. 3C depicts the MIC values for the humimycins against a panel of human commensal and pathogenic bacteria (n=3). FIG. 3D depicts the activity of the humimycins against bacteria in the Staphylococcus and Streptococcus genus (n=3). FIG. 3E depicts the human microbiota composition at different sites of the body (D'Argenio and Salvatore, Clin Chim Acta 2015, 451:97-102).

FIG. 4 is a table providing the MICs of humimycin A alanine scan analogs. The superscripts in the table are as follows:^{a} indicates that the Minimal inhibitory concentration (MIC) values are expressed in units of μg/mL;^{b} indicates that “>” denotes MIC values greater than 128 μg/mL, the highest concentration tested;^{c} indicates that alanine substitution analogs were synthesized on 2-chlorotrityl resins, the first amino acid (1.2 equiv.) was loaded in the presence of DIEA (3 equiv.) and capped by DCM/MeOH/DIEA (80:15:5) treatment for 30 min, the rest of the SPPS steps were identical to those described in the peptide synthesis section of the methods of Example 1;^{d} indicates that HMA denotes N-terminal modification with 3-hydroxymyrixtic acid; 3 indicates that amino acids are abbreviated using standard 1-letter codes, with upper and lower case letters denoting amino acids in L- and D-forms respectively; and^{e} indicates that humimycin A (1) refers to the syn-BNP N-acylated with the (S)-3-hydroxymyristic acid (FIG. 6).

FIG. 5, comprising FIGS. 5A to 5D, depicts an analysis of susceptibility and resistance to humimycin A. FIG. 5A depicts clinical isolates of S. aureus that are susceptible to humimycin A (n=2). FIG. 5B depicts that humimycin A resistance mutations cluster in the first 7 transmembrane helices of SAV1754 (see FIG. 8 for details). FIG. 5C depicts a schematic diagram for one possible mode of action of humimycin A, inhibiting SAV1754, the S. aureus homolog of MurJ, a flippase responsible for the transportation of peptidoglycan precursors across the cytoplasmic membrane. FIG. 5D depicts that carbenicillin (C) and humimycin A (H) act synergistically to inhibit the growth of MRSA USA300 (n=3; FIG. 9). Fraction inhibitory concentration (FIC) values ≤0.5 defines synergy between two agents (shaded in light gray); [C;H] denotes the respective concentrations at each data point (μg/mL).

FIG. 6, comprising FIGS. 6A to 6E, depicts HPLC traces used to assign diastereomers of humimycin A. FIGS. 6A and 6B depict HPLC traces of small scale syntheses of humimycin A using enantiopure (R) and (S)-3-hydroxymyristic acid, respectively. FIGS. 6C and 6D depict reinjection of HPLC-purified humimycin A diastereomers from bulk syntheses. Humimycin A (1) refers to the peptide N-acylated with the (S)-3-hydroxymyristic acid (d) that shows more potent antibiotic activity (FIG. 4). HPLC runs were performed on an Agilent Technologies instrument (1200 series) with detection at 220 nm. FIG. 6E depicts using the gradient used for performing the HPLC runs. The flow rate was 4 mL/min through a reverse-phase column (XBridge BEH C18, 130 Å, 5 um, 4.6×150 mm, Waters Corporation). Mobile phases A and B were water and acetonitrile with 0.1% TFA, respectively.

FIG. 7, comprising FIGS. 7A and 7B, is a set of tables providing bacterial strain information for those strains evaluated. FIG. 7A depicts pathogenic bacteria. FIG. 7B depicts commensal bacteria. The superscripts in the table identify special growth conditions as follows: * indicates anaerobic atmosphere; and indicates 30° C. incubation. LB and BHI media were prepared directly from premixed powder purchased from BD Biosciences. The LYBHI medium contains (per liter) BHI (37 g), yeast extract (5 g), maltose (1 g), cellobiose (1 g), cysteine (0.5 g), and hemin (5 mg).

FIG. 8 is a table presenting the SNPs found in MRSA USA300 strains that were identified as resistant to humimycin A. Among 23 fully sequenced mutants, 22 acquired a single point mutation on protein SAV1754 and 1 acquired two mutations. A number of these mutants (9) acquired mutations, in addition to SAV1754, located elsewhere in the genome.

FIG. 9, comprising FIGS. 9A to 9C, depicts the raw data of the carbenicillin-humimycin A synergy assays. FIGS. 9A, 9B, and 9C depict individual replicates of the synergy assay of carbenicillin and humimycin A. Synergy data is not scaled to their respective fraction inhibitory concentrations (FIC).

### DETAILED DESCRIPTION

The present invention relates to the surprising and unexpected discovery of compounds identified using an approach that circumvents the need for either bacterial culture or gene cluster expression. For example, the compounds can be identified using a bioinformatic predicted methodology and produced by chemical synthesis. Accordingly, in certain instances the methodology is referred to herein as Synthetic-Bioinformatic Natural Product (syn-BNPs). In one embodiment of the invention, the compounds are bioinformatically predicted from primary sequence data and produced by chemical synthesis.

In one embodiment, the present invention provides compounds or a therapeutic compound comprising a desired activity. In one embodiment, the compound is an antibiotic. In embodiment, the antibiotic compound of the invention can be used in the treatment of bacterial infections. In certain embodiments, the use of the antibiotic compound of the invention in the treatment of bacterial infections optionally includes a pharmaceutically acceptable carrier, excipient or adjuvant.

In one embodiment, the compounds of the invention include hepta-peptide antibiotics, referred elsewhere herein as humimycin A and B. However, the invention should not be limited to these compounds. Rather, the invention encompasses any compound that is bioinformatically predicted from primary sequence data and produced by chemical synthesis using the methods of the invention.

In one embodiment, the compound is an isolated nucleic acid, isolated peptide, small molecule, peptidomimetic, or the like as well as any derivative or analog thereof.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

An “amino terminus modification group” refers to any molecule that can be attached to the amino terminus of a polypeptide. Similarly, a “carboxy terminus modification group” refers to any molecule that can be attached to the carboxy terminus of a polypeptide. Terminus modification groups include but are not limited to various water soluble polymers, peptides or proteins such as serum albumin, or other moieties that increase serum half-life of peptides.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The term, “biologically active” or “bioactive” can mean, but is in no way limited to, the ability of an agent or compound to effectuate a physiological change or response. The response may be detected, for example, at the cellular level, for example, as a change in growth and/or viability, gene expression, protein quantity, protein modification, protein activity, or combination thereof; at the tissue level; at the systemic level; or at the organism level. For example, as used herein, biologically active molecules include but are not limited to any substance intended for diagnosis, cure, mitigation, treatment, or prevention of disease in humans or other animals, or to otherwise enhance physical or mental well-being of humans or animals. Examples of biologically active molecules include, but are not limited to, peptides, proteins, enzymes, small molecule drugs, dyes, lipids, nucleosides, oligonucleotides, cells, viruses, liposomes, microparticles and micelles. Classes of biologically active agents that are suitable for use with the invention include, but are not limited to, antibiotics, fungicides, anti-viral agents, anti-inflammatory agents, anti-tumor agents, cardiovascular agents, anti-anxiety agents, hormones, growth factors, steroidal agents, and the like.

The term “conservative mutations” refers to the substitution, deletion or addition of nucleic acids that alter, add or delete a single amino acid or a small number of amino acids in a coding sequence where the nucleic acid alterations result in the substitution of a chemically similar amino acid. Amino acids that may serve as conservative substitutions for each other include the following:
Basic: Arginine (R), Lysine (K), Histidine (H);
Acidic: Aspartic acid (D), Glutamic acid (E);
Neutral: Asparagine (N), Cysteine (C), Glutamine (Q), Methionine (M), Serine (S), Threonine (T);
Aliphatic: Alanine (A), Valine (V), Leucine (L), Isoleucine (I), Glycine (G);
Hydrophobic—Aromatic: Phenylalanine (F), Tyrosine (Y), Tryptophan (W);
Sulfur-containing: Methionine (M), Cysteine (C)
Hydroxyl: Serine (S), Threonine (T);
Aminde: Asparagine (N), Glutamine (Q).

In addition, sequences that differ by conservative variations are generally homologous. In some instances, the following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

As used herein, “derivatives” are compositions formed from the native compounds either directly, by modification, or by partial substitution. As used herein, “analogs” are compositions that have a structure similar to, but not identical to, the native compound.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

In the context of the invention, term “natural amino acid” means any amino acid which is found naturally in vivo in a living being. Natural amino acids therefore include amino acids coded by mRNA incorporated into proteins during translation but also other amino acids found naturally in vivo which are a product or by-product of a metabolic process, such as for example ornithine which is generated by the urea production process by arginase from L-arginine. In the invention, the amino acids used can therefore be natural or not. Namely, natural amino acids generally have the L configuration but also, according to the invention, an amino acid can have the L or D configuration.

A “non-naturally encoded amino acid” refers to an amino acid that is not one of the 20 common amino acids or pyrolysine or selenocysteine. The term “non-naturally encoded amino acid” includes, but is not limited to, amino acids that occur naturally by modification of a naturally encoded amino acid (including but not limited to, the 20 common amino acids or pyrolysine and selenocysteine) but are not themselves incorporated into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Parenteral” administration of a composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. Furthermore, peptides of the invention may include amino acid mimentics, and analogs. Recombinant forms of the peptides can be produced according to standard methods and protocols which are well known to those of skill in the art, including for example, expression of recombinant proteins in prokaryotic and/or eukaryotic cells followed by one or more isolation and purification steps, and/or chemically synthesizing peptides or portions thereof using a peptide sythesizer.

Conventional notation is used herein to portray polypeptide sequences: the left-hand end of a polypeptide sequence is the amino-terminus; the right-hand end of a polypeptide sequence is the carboxyl-terminus.

As used herein, a “peptidomimetic” is a compound containing non-peptidic structural elements that is capable of mimicking the biological action of a parent peptide. A peptidomimetic may or may not comprise peptide bonds.

The term “recombinant polypeptide” as used herein is defined as a polypeptide produced by using recombinant DNA methods. A host cell that comprises a recombinant polynucleotide is referred to as a “recombinant host cell.” A gene which is expressed in a recombinant host cell wherein the gene comprises a recombinant polynucleotide, produces a “recombinant polypeptide.”

The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration.

Non-limiting examples of agents suitable for formulation with the, e.g., compounds provided by the instant invention include: cinnamoyl, PEG, phospholipids or lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The term “pharmaceutically acceptable” or “pharmacologically acceptable” can mean, but is in no way limited to, entities and compositions that do not produce an adverse, allergic or other untoward reaction when administered to an animal, or a human, as appropriate.

The term “pharmaceutically acceptable carrier” or “pharmacologically acceptable carrier” can mean, but is in no way limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

As used herein, the term “alkyl,” by itself or as part of another substituent means, unless otherwise stated, a straight or branched chain hydrocarbon having the number of carbon atoms designated (i.e. C₁-₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, particularly ethyl, methyl, isopropyl, isobutyl, n-pentyl, n-hexyl and cyclopropylmethyl.

As used herein, the term “substituted alkyl” means alkyl as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

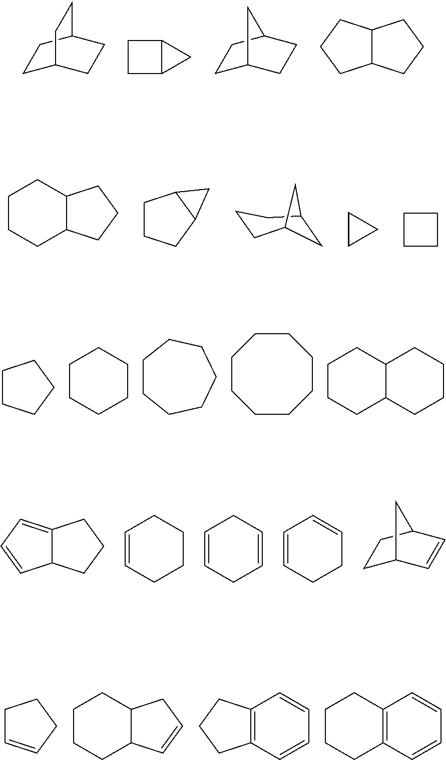

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

In one aspect, the present invention relates to a bioinformatic method of predicting a desired compound based on genetic information and the chemical synthesis of the predicted compound. In certain embodiment, the desired compound is an antibiotic. In certain embodiments, the compound comprises an amino acid sequence of YSY[X₁]T[X₂]V or any derivative or analog thereof.

In one embodiment, the invention provides a compound exhibiting a desired biological activity whereby the compound is identified and produced without the need for either bacterial culture or gene cluster expression. For example, a compound having a desired biological activity can be predicted using a bioinformatic methodology and produced by chemical synthesis. In one embodiment, a bioinformatic methodology for predicting a compound uses primary sequencing data or genomic information. Therefore, in various embodiments, the invention relates to a method of predicting a compound based on genomic information and optionally further chemically synthesizing the predicted compound; and also relates to a compound identified and synthesized using the methods of the invention.

In one embodiment, a desired biological activity of a compound of the invention is an antibiotic. Therefore, the invention provides a method for predicting an antibiotic compound from genomic data as well as novel antibiotic compounds. In one embodiment, the invention also relates to a method for treating a disease or condition using the compounds of the invention. However, the invention should not be limited to predicting an antibiotic. Rather, the invention includes using a bioinformatics methodology is predict a compound having a desired activity.

### Method of Predicting a Compound

The field of bioinformatics involves the collection, classification, storage, and analysis of biochemical and biological information using computers. One of the most broadly used bioinformatics approaches is analysis of genomic information. Accordingly, it is an object of the present invention to provide a bioinformatic method of predicting compounds from a microorganism, such as bacteria, which can be chemically synthesized and utilized in methods of treating or preventing infectious diseases arising from the microorganism or equivalent thereof.

In one embodiment, the present invention relates to a method for rapid identification and analysis of modular biosynthetic gene-clusters starting from DNA sequences for the generation of novel biologically active chemical entities, the method comprising the steps of: a) evaluating genomic DNA information from one or more specie of interest to identify potential gene clusters; b) evaluating identified gene clusters to determine the module or domain content; c) predicting one or more compounds produced by the gene cluster(s); and d) selecting one or more predicted compounds for chemical synthesis.

In one embodiment, potential gene clusters from genomic DNA and the modules or domains of these gene clusters can be predicted using bioinformatic programs. The predictions are used to deduce the chemical structure of the products produced by the catalytic domains encoded by the gene-cluster and the results are evaluated to predict the biological compound. The predicted biological compound(s) are then chemically synthesized. The method also allows the generation of a library of predicted chemical compounds.

In the general process of the invention, the method comprises searching genomic sequence information on one or more microorganism such as bacteria. In one embodiment, genomic sequence information is obtained from a database containing such information. In one embodiment, a database containing bacterial genomic sequence information is the NIH Human Microbiome Project (HMP). In one embodiment, a database containing bacterial genomic sequence information is the Human Oral Microbiome Database (HOMD). In one embodiment, a database may be a public access database. In one embodiment, a database may be a restricted access database. In alternative embodiments, genomic sequence information may be obtained directly from sequencing, from an individual researcher or from a collaboration.

The bioinformatics method of the invention relates to identifying gene clusters, and predicting the compounds produced therefrom. In one embodiment, the method relates to identifying one or more of PKS, NRPS, NIS and hybrid synthase/synthetase gene-clusters. In one embodiment, an algorithm, module (e.g. bioperl module) or computer program for predicting gene clusters from genomic data is used. For example, Antibiotics and Secondary Metabolite Analysis Shell (antiSMASH) v2.0, 2metDB, SMIPS, SEARCHPKS, CLUSEAN, ClusterFinder, eSNaPD (environmental Surveyor of Natural Product Diversity), BAGEL, CASSIS, ClustScan Professional, EvoMining, FunGeneClusterS, GNP/PRISM, MIDDAS-M, MIPS-CG, NaPDoS (Natural Products Domain Seeker), SMURF (Secondary Metabolite Unknown Region Finder) ClusterMine360, NP.searcher, NRPSpredictor2, and NRPSsp are appropriate algorithms, computer programs, or modules for use for use in predicting gene clusters.

In one embodiment, a predicted gene cluster of the invention is a NRP gene cluster. In one embodiment, a predicted NRP gene cluster includes an adenylation domain; a PCP domain (thiolation and peptide carrier protein domain) and a condensation domain. In one embodiment, a predicted NRP gene cluster further includes one or more of a formylation (F) domain; a cyclization (Cy) domain; an oxidation (OX) domain; a reduction (Red) domain; an epimerization (E) domain, an N-methylation (NMT) domain, a termination by a thio-esterase (TE) domain and a reduction to terminal aldehyde or alcohol (R) domain.

In one embodiment, a predicted gene cluster of the invention is a PKS gene cluster. In one embodiment, a predicted PKS gene cluster includes one or more of an acyltransferase (AT) domain; an acyl carrier protein (ACP) domain; a keto-synthase (KS) domain; a ketoreductase (KR) domain; a dehydratase (DH) domain; an enoylreductase (ER) domain; a methyltrasferase (MT) domain; a sulfhydrolase (SH) domain; and a thioesterase (TE) domain.

In one embodiment, a predicted gene cluster is a hybrid PKS/NRPS gene cluster. A predicted hybrid PKS/NRPS gene cluster may contain PKS modules/domains in addition to NRP modules/domains.

A further aspect of the method of predicting a compound produced from a gene cluster relates to the use of one or more algorithms, computer programs, or modules (e.g. a bioperl module) to predict the compound. Algorithms, computer programs, or modules appropriate for use in predicting a compound produced by a gene cluster include but are not limited to GNP/PRISM, BAGEL, CASSIS, Stachelhaus, Minowa, NRPS-PKS and NRPSPredictor2. In one embodiment, a combination of algorithms for predicting the compound produced by an NRP of the invention includes Stachelhaus, Minowa, and NRPSPredictor2. In one embodiment, a multitude of algorithms for predicting the compound produced by a gene cluster all provide the same prediction, or a ‘consensus’ prediction, and the consensus predicted compound is selected for chemical synthesis. In one embodiment, a multitude of algorithms for predicting the compound produced by a gene cluster provide different predictions. In one embodiment, different predictions from a multitude of algorithms are evaluated to be minor, and a compound selected for chemical synthesis is selected to contain the predicted amino acid with the smaller side-chain from the differently predicted compounds. In one embodiment, different predictions from a multitude of algorithms are evaluated to be due to known biases in one or more of the prediction algorithms, and a compound selected for chemical synthesis is selected to compensate for the known bias. In one embodiment, different predictions from a multitude of algorithms are evaluated to provide peptides having differentially charged side chains, and a multitude of compounds are selected for chemical synthesis based on the different predictions.

In one embodiment, a predicted NRP gene cluster of the invention is further predicted to produce a compound having fewer than 5 residues. In one embodiment, a predicted NRP gene cluster of the invention is further predicted to produce a compound having at least 5 residues. In one embodiment, a predicted NRP gene cluster of the invention is further predicted to produce a cyclic compound. In one embodiment, a predicted NRP gene cluster of the invention is further predicted to produce a linear compound. In one embodiment, a predicted NRP gene cluster of the invention is further predicted to produce a methylated compound. In one embodiment, a predicted NRP gene cluster of the invention is further predicted to produce an alkylated compound. In one embodiment, a predicted NRP gene cluster of the invention is further predicted to produce a compound having one of D-, L- and both D- and L-amino acids.

In one embodiment, a single compound is selected for synthesis based on the bioinformatics prediction method. In one embodiment, multiple compounds are selected for synthesis based on the bioinformatics prediction method. In one embodiment, multiple compounds selected for synthesis are at least two compounds having different amino acid side chains. In one embodiment, multiple compounds selected for synthesis have the same amino acid sequence but have different modifications. Without being limiting, in one embodiment, a modification can be one of methylation, adenylation, acetylation, alkylation, formylation, cyclization, and epimerization.

### Compositions

In one aspect, the present invention provides compositions having a desired activity. For example, the composition of the invention is an antibiotic. The compositions, may be used, for example, to inhibit bacterial growth. Exemplary compounds, include, but are not limited to, isolated peptides, peptide mimetics, small molecules, and the like.

In one embodiment, the composition of the present invention comprises a compound comprising peptide having an amino acid sequence of YSY[X₁]T[X₂]V or a biologically functional derivative or analog thereof. In certain embodiments, the amino acid sequence is selected from YSY[X₁]T[X₂]V (SEQ ID NO: 1), YSYFTVV (SEQ ID NO: 2), YSYYTIV (SEQ ID NO: 3), and any combination thereof.

In one embodiment, X₁ is F or Y. In one embodiment, X₁ can be F, Y, or W. In one embodiment, X₁ can be any amino acid that maintains the biological activity of the peptide. In one embodiment, X₁ can be A, N, C, Q, G, I, L, M, F, P, S, T, W, Y, or V.

In one embodiment, X₂ is V or I. In one embodiment, X₂ is A, V, L, I, or G. In one embodiment, X2 can be any amino acid that maintains the biological activity of the peptide. In one embodiment, X2 can be A, N, C, Q, G, I, L, M, F, P, S, T, W, Y, or V.

The peptide of interest can contain at least one, at least two, at least three, at least four, at least five, at least six, at least seven, at least eight, at least nine, or ten or more non-natural amino acids. The unnatural amino acids can be the same or different, for example, there can be 1, 2, 3, 4, 5, 6, 7, 8, 9, 10 or more different sites in the protein that comprise 1, 2, 3, 4, 5, 6, 7, 8, 9, 10 or more different unnatural amino acids. In certain embodiments, at least one, but fewer than all, of a particular amino acid present in a naturally occurring version of the protein is substituted with an unnatural amino acid.

The compounds present in the compositions of the invention may or not be optically pure, which means that the lysine residues in the peptide units may either be in random L or D configuration (not optically pure), or be all in D configuration or all in L configuration (optically pure).

In one embodiment, the peptides of the invention comprises an N-terminal modification. In one embodiment, the N-terminal modification comprises an acyl group on the N-terminus of the peptide. In one embodiment, the N-terminal modification comprises a fatty acid on the N-terminus of the peptide. In one embodiment, the fatty acid is β-hydroxymyristic acid (HMA).

In one embodiment, the compositions of the invention also encompasses peptides having minor modifications, for example, conservative amino acid modifications, chemical modification to mimic valence properties, and modifications that serve to increase its stability, solubility, biouptake and/or bioavailability. A pharmaceutically acceptable carrier can contain physiologically acceptable compounds that include carbohydrates such as glucose, sucrose or dextrans; antioxidants, such as ascorbic acid or glutathione; chelating agents; and low molecular weight proteins. Additional modifications to the compound of the invention that can increase its bioavailability include conjugating the peptide to a lipophilic moiety, such as a lipophilic amino acid or compound.

The compound of the invention is further intended to encompass peptides bearing one or several minor modifications to the amino acid sequence. Contemplated modifications include chemical or enzymatic modifications (e.g. acylation, phosphorylation, glycosylation, etc.), and substitutions of one or several amino acids to the peptide sequence. Those skilled in the art recognize that such modifications can be desirable in order to enhance the bioactivity, bioavailability or stability of the peptide, or to facilitate its synthesis or purification.

Contemplated amino acid substitutions to the compounds provided by the invention, include conservative changes, wherein a substituted amino acid has similar structural or chemical properties (e.g., replacement of an apolar amino acid with another apolar amino acid; replacement of a charged amino acid with a similarly charged amino acid, etc.). Those skilled in the art also recognize that nonconservative changes (e.g., replacement of an uncharged polar amino acid with an apolar amino acid; replacement of a charged amino acid with an uncharged polar amino acid, etc.) can be made without affecting the function of the compound. Furthermore, non-linear variants of the peptide sequence, including branched sequences and cyclic sequences, and variants that contain one or more D-amino acid residues in place of their L-amino acid counterparts, may be made.

In one embodiment, the invention includes variants of the peptides of the invention. The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one polypeptide to a sequence of a second polypeptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence and/or the ability to stimulate the differentiation of a stem cell into the osteoblast lineage. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery.

The peptides of the invention may include unnatural amino acids formed by post-translational modification or by introducing unnatural amino acids during translation. A variety of approaches are available for introducing unnatural amino acids during protein translation.

A very wide variety of non-naturally encoded amino acids are suitable for use in the present invention. Any number of non-naturally encoded amino acids can be introduced into the peptide if the invention. In general, the introduced non-naturally encoded amino acids are substantially chemically inert toward the 20 common, genetically-encoded amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). In some embodiments, the non-naturally encoded amino acids include side chain functional groups that react efficiently and selectively with functional groups not found in the 20 common amino acids (including but not limited to, azido, ketone, aldehyde and aminooxy groups) to form stable conjugates. For example, a peptide of the invention that includes a non-naturally encoded amino acid containing an azido functional group can be reacted with a polymer (including but not limited to, poly(ethylene glycol) or, alternatively, a second polypeptide containing an alkyne moiety to form a stable conjugate resulting for the selective reaction of the azide and the alkyne functional groups to form a Huisgen [3+2] cycloaddition product.

In some instances, the non-naturally encoded amino acids of the invention typically differ from the natural amino acids only in the structure of the side chain. The non-naturally encoded amino acids form amide bonds with other amino acids, including but not limited to, natural or non-naturally encoded, in the same manner in which they are formed in naturally occurring polypeptides. However, the non-naturally encoded amino acids have side chain groups that distinguish them from the natural amino acids. For example, R optionally comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amino group, or the like or any combination thereof. Other non-naturally occurring amino acids of interest that may be suitable for use in the present invention include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or noncovalently interact with other molecules, photocaged and/or photoisomerizable amino acids, amino acids comprising biotin or a biotin analogue, glycosylated amino acids such as a sugar substituted serine, other carbohydrate modified amino acids, keto-containing amino acids, amino acids comprising polyethylene glycol or polyether, heavy atom substituted amino acids, chemically cleavable and/or photocleavable amino acids, amino acids with an elongated side chains as compared to natural amino acids, including but not limited to, polyethers or long chain hydrocarbons, including but not limited to, greater than about 5 or greater than about 10 carbons, carbon-linked sugar-containing amino acids, redox-active amino acids, amino thioacid containing amino acids, and amino acids comprising one or more toxic moiety.

Many unnatural amino acids are based on natural amino acids, such as tyrosine, glutamine, phenylalanine, and the like, and are suitable for use in the present invention. Tyrosine analogs include, but are not limited to, para-substituted tyrosines, ortho-substituted tyrosines, and meta substituted tyrosines, where the substituted tyrosine comprises, including but not limited to, a keto group (including but not limited to, an acetyl group), a benzoyl group, an amino group, a hydrazine, an hydroxyamine, a thiol group, a carboxy group, an isopropyl group, a methyl group, a C₆-C₂₀ straight chain or branched hydrocarbon, a saturated or unsaturated hydrocarbon, an O-methyl group, a polyether group, a nitro group, an alkynyl group or the like. In addition, multiply substituted aryl rings are also contemplated. Glutamine analogs that may be suitable for use in the present invention include, but are not limited to, alpha-hydroxy derivatives, gamma-substituted derivatives, cyclic derivatives, and amide substituted glutamine derivatives. Example phenylalanine analogs that may be suitable for use in the present invention include, but are not limited to, para-substituted phenylalanines, ortho-substituted phenyalanines, and meta-substituted phenylalanines, where the substituent comprises, including but not limited to, a hydroxy group, a methoxy group, a methyl group, an allyl group, an aldehyde, an azido, an iodo, a bromo, a keto group (including but not limited to, an acetyl group), a benzoyl, an alkynyl group, or the like. Specific examples of unnatural amino acids that may be suitable for use in the present invention include, but are not limited to, a p-acetyl-L-phenylalanine, an O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAc.beta.-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, and a p-propargyloxy-phenylalanine, and the like.

Non-naturally encoded amino acid polypeptides presented herein may include isotopically-labelled compounds with one or more atoms replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as ²H, ³H, ¹³C, ¹⁴C, ¹⁵N, ¹⁸O, ¹⁷O, ³⁵S, ¹⁸F, ³⁶Cl, respectively. Certain isotopically-labelled compounds described herein, for example those into which radioactive isotopes such as ³H and ¹⁴C are incorporated, may be useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements.

All isomers including but not limited to diastereomers, enantiomers, and mixtures thereof are considered as part of the compositions described herein. In additional or further embodiments, the non-naturally encoded amino acid polypeptides are metabolized upon administration to an organism in need to produce a metabolite that is then used to produce a desired effect, including a desired therapeutic effect. In further or additional embodiments are active metabolites of non-naturally encoded amino acid polypeptides.

In some situations, non-naturally encoded amino acid polypeptides may exist as tautomers. In addition, the non-naturally encoded amino acid polypeptides described herein can exist in unsolvated as well as solvated forms with pharmaceutically acceptable solvents such as water, ethanol, and the like. The solvated forms are also considered to be disclosed herein. Those of ordinary skill in the art will recognize that some of the compounds herein can exist in several tautomeric forms. All such tautomeric forms are considered as part of the compositions described herein.

The ability to incorporate non-genetically encoded amino acids into proteins permits the introduction of chemical functional groups that could provide valuable alternatives to the naturally-occurring functional groups, such as the epsilon —NH₂ of lysine, the sulfhydryl —SH of cysteine, the imino group of histidine, etc. Certain chemical functional groups are known to be inert to the functional groups found in the 20 common, genetically-encoded amino acids but react cleanly and efficiently to form stable linkages. Azide and acetylene groups, for example, are known in the art to undergo a Huisgen[3+2] cycloaddition reaction in aqueous conditions in the presence of a catalytic amount of copper. See, e.g., Tornoe, et al., (2002) J. Org. Chem. 67:3057-3064; and, Rostovtsev, et al., (2002) Angew. Chem. Int. Ed. 41:2596-2599. By introducing an azide moiety into a protein structure, for example, one is able to incorporate a functional group that is chemically inert to amines, sulfhydryls, carboxylic acids, hydroxyl groups found in proteins, but that also reacts smoothly and efficiently with an acetylene moiety to form a cycloaddition product. Importantly, in the absence of the acetylene moiety, the azide remains chemically inert and unreactive in the presence of other protein side chains and under physiological conditions.

A peptide or protein of the invention may be conjugated with other molecules, such as proteins, to prepare fusion proteins. This may be accomplished, for example, by the synthesis of N-terminal or C-terminal fusion proteins provided that the resulting fusion protein retains the functionality the peptide of the invention.

A peptide or protein of the invention may be phosphorylated using conventional methods such as the method described in Reedijk et al. (The EMBO Journal 11(4):1365, 1992).

Cyclic derivatives of the peptides of the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The invention also relates to peptides comprising peptides of the invention fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e., are heterologous).

A peptide of the invention may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a peptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

The peptides described herein can be produced using well known recombinant methods or via well-known synthetic methods. There are several well-known methods for performing peptide synthesis including liquid-phase and solid-phase synthesis. Detailed discussions of various methods can be found at, for example, Atherton, E.; Sheppard, R. C. (1989). Solid Phase peptide synthesis: a practical approach. Oxford, England: IRL Press; Stewart, J. M.; Young, J. D. (1984). Solid phase peptide synthesis, 2nd edition, Rockford: Pierce Chemical Company, 91; R. B. Merrifield (1963). “Solid Phase Peptide Synthesis. I. The Synthesis of a Tetrapeptide”. J. Am. Chem. Soc. 85 (14): 2149-2154; L. A. Carpino (1993). “1-Hydroxy-7-azabenzotriazole. An efficient peptide coupling additive”. J. Am. Chem. Soc. 115 (10): 4397-4398; which are hereby incorporated by reference in their entirety for all purposes.

N-terminal or C-terminal fusion proteins comprising a peptide or chimeric protein of the invention conjugated with other molecules may be prepared by fusing, through recombinant techniques, the N-terminal or C-terminal of the peptide or chimeric protein, and the sequence of a selected protein or selectable marker with a desired biological function. The resultant fusion proteins contain the peptide of the invention fused to the selected protein or marker protein as described herein. Examples of proteins which may be used to prepare fusion proteins include immunoglobulins, glutathione-S-transferase (GST), hemagglutinin (HA), and truncated myc.

The peptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

The compounds of the invention including derivatives or analogues thereof are also easily synthesized, even on an industrial scale, under easily controllable health safety conditions.

### Nucleic Acid

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The isolated nucleic acid may comprise any type of nucleic acid, including, but not limited to DNA and RNA. For example, in one embodiment, the composition comprises an isolated DNA molecule, including for example, an isolated cDNA molecule, encoding the peptide. In one embodiment, the composition comprises an isolated RNA molecule encoding the peptide.

The desired polynucleotide can be cloned into a number of types of vectors. However, the present invention should not be construed to be limited to any particular vector. Instead, the present invention should be construed to encompass a wide plethora of vectors which are readily available and/or well-known in the art. For example, a desired polynucleotide of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In one embodiment, the present invention provides a composition comprising an isolated nucleic acid encoding the peptide of the invention, or a biologically functional derivative or analog thereof. In one embodiment, the isolated nucleic acid sequence encodes the peptide of the invention, or a biologically functional derivative or analog thereof comprises an amino acid sequence selected from YSY[X₁]T[X₂]V (SEQ ID NO: 1), YSYFTVV (SEQ ID NO: 2), YSYYTIV (SEQ ID NO: 3), and any combination thereof.

Further, the invention encompasses an isolated nucleic acid encoding a peptide having substantial homology to the peptides disclosed herein. In certain embodiments, the isolated nucleic acid sequence encodes the peptide of the invention, or a biologically functional derivative or analog thereof having at least 75%, 80%, 85%, 90%, 95%, 96%, 97%, 98%, or 99% sequence homology with an amino acid sequence selected from SEQ NOs: 1-3.

The isolated nucleic acid sequence encoding the peptide of the invention, or a biologically functional derivative or analog thereof can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The isolated nucleic acid may comprise any type of nucleic acid, including, but not limited to DNA and RNA. For example, in one embodiment, the composition comprises an isolated DNA molecule, including for example, an isolated cDNA molecule, encoding the peptide of the invention, or a biologically functional derivative or analog thereof. In one embodiment, the composition comprises an isolated RNA molecule encoding the peptide of the invention, or a biologically functional derivative or analog thereof.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In some instances, the nucleic acid molecule comprises at least one of the following chemical modifications: 2′-H, 2′-O-methyl, or 2′-OH modification of one or more nucleotides. In certain embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

### Treatment Methods

When used as antibiotic compounds for the treatment of a bacterial infection, the compounds of the invention are preferably in a therapeutic composition comprising an effective amount of the desired compound of the invention. However, the invention should not be limited to only treating bacterial infection. The invention encompasses compounds having an antimicrobial activity including but not limited to antibacterial, antimycobacterial, antifungal, antiviral and the likes.

In any of the compositions described herein, the therapeutic compositions provided by the invention optionally includes a pharmaceutically acceptable carrier, excipient or adjuvant.

In another aspect, the invention provides compositions and methods for treating and/or preventing a disease or disorder related to the detrimental growth and/or proliferation of a bacterial cell in vivo, ex vivo or in vitro. In certain embodiments, the method comprises administering a composition comprising an effective amount of a composition provided by the invention to a subject, wherein the composition is effective in inhibiting or preventing the growth and/or proliferation of a bacterial cell. In certain embodiments, the bacterial cell is a Gram+ bacterial cell, e.g., a bacteria of a genera such as Staphylococcus, Streptococcus, Enterococcus, (which are cocci) and Bacillus, Corynebacterium, Nocardia, Clostridium, Actinobacteria, and Listeria (which are rods and can be remembered by the mnemonic obconical), Mollicutes, bacteria-like Mycoplasma, Actinobacteria.

In certain embodiments, the bacterial cell is a Gram− bacteria cell, e.g., a bacteria of a genera such as Citrobacter, Yersinia, Pseudomonas and Escherichia, Hemophilus, Neisseria, Klebsiella, Legionella, Helicobacter, and Salmonella. The compounds as described herein and compositions comprising them may thus be for use in the treatment of bacterial infections by the above mentioned Gram+ or Gram− bacteria.

As illustrated in the examples, humimycin compounds have been found to display antibiotic activity against Staphylococcus and Streptococcus genus, including common members of the normal human flora such as S. aureus and S. pneumoniae, and MRSA clinical isolates. The compositions and methods of the invention may thus advantageously be used for the treatment of infections caused by one of the bacteria species discussed elsewhere herein.

Notably, S. aureus can cause a range of illnesses from minor skin infections, such as pimples, impetigo, boils (furuncles), cellulitis folliculitis, carbuncles, scalded skin syndrome, and abscesses, to life-threatening diseases such as pneumonia, meningitis, osteomyelitis, endocarditis, toxic shock syndrome (TSS), bacteremia, and sepsis. Its incidence is from skin, soft tissue, respiratory, bone, joint, endovascular to wound infections. It is still one of the five most common causes of nosocomial infections, often causing postsurgical wound infections. Each year, some 500,000 patients in American hospitals contract a staphylococcal infection. The humimycin compounds or compositions comprising them may thus notably be for use as antibiotic drug in the treatment of Staphylococcus aureus infections.

The present invention relates to the unexpected discovery that a humimycin compound acts as an antimicrobial agent. In one embodiment, a humimycin compound or the analog thereof has an antimicrobial activity selected from the group consisting of antibacterial, antimycobacterial, antifungal, antiviral and any combinations thereof.

A humimycin compound or an analog thereof may be used, alone or in combination with at least one additional antimicrobial agent. In one embodiment, a humimycin compound or an analog thereof and the at least one additional antimicrobial agent act synergistically in preventing, reducing or disrupting microbial growth. Non-limiting examples of the at least one additional antimicrobial agent are levofloxacin, doxycycline, neomycin, clindamycin, minocycline, gentamycin, rifampin, chlorhexidine, chloroxylenol, methylisothizolone, thymol, α-terpineol, cetylpyridinium chloride, hexachlorophene, triclosan, nitrofurantoin, erythromycin, nafcillin, cefazolin, imipenem, astreonam, gentamicin, sulfamethoxazole, vancomycin, ciprofloxacin, trimethoprim, rifampin, metronidazole, clindamycin, teicoplanin, mupirocin, azithromycin, clarithromycin, ofoxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linexolid, temafloxacin, tosufloxacin, clinafloxacin, sulbactam, clavulanic acid, amphotericin B, fluconazole, itraconazole, ketoconazole, nystatin, penicillins, cephalosporins, carbepenems, beta-lactams antibiotics, aminoglycosides, macrolides, lincosamides, glycopeptides, tetracylines, chloramphenicol, quinolones, fucidines, sulfonamides, trimethoprims, rifamycins, oxalines, streptogramins, lipopeptides, ketolides, polyenes, azoles, echinocandines, and any combination thereof.

In one embodiment, the compositions of the invention find use in removing at least a portion of or reducing the number of microorganisms and/or biofilm-embedded microorganisms attached to the surface of a medical device or the surface of a subject's body (such as the skin of the subject, or a mucous membrane of the subject, such as the vagina, anus, throat, eyes or ears). In one embodiment, the compositions of the invention find further use in coating the surface of a medical device, thus inhibiting or disrupting microbial growth and/or inhibiting or disrupting the formation of biofilm on the surface of the medical device. The compositions of the invention find further use in preventing or reducing the growth or proliferation of microorganisms and/or biofilm-embedded microorganisms on the surface of a medical device or on the surface of a subject's body. However, the invention is not limited to applications in the medical field. Rather, the invention includes using a humimycin compound or an analog thereof as an antimicrobial and/or antibiofilm agent in any setting.

The composition of the invention may be administered to a patient or subject in need in a wide variety of ways, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the composition is administered systemically to the subject. In one embodiment, the compositions of the present invention are administered to a patient by i.v. injection. In one embodiment, the composition is administered locally to the subject. In one embodiment, the compositions of the present invention are administered to a patient topically. Any administration may be a single application of a composition of invention or multiple applications. Administrations may be to single site or to more than one site in the individual to be treated. Multiple administrations may occur essentially at the same time or separated in time.

In one aspect, the compositions of the invention may be in the form of a coating that is applied to the surface of a medical device or the surface of a subject's body. In one embodiment, the coating prevents or hinders microorganisms and/or biofilm-embedded microorganisms from growing and proliferating on at least one surface of the medical device or at least one surface of the subject's body. In another embodiment, the coating facilitates access of antimicrobial agents to the microorganisms and/or biofilm-embedded microorganisms, thus helping prevent or hinder the microorganisms and/or biofilm-embedded microorganisms from growing or proliferating on at least one surface of the medical device or at least one surface of the subject's body. The compositions of the invention may also be in the form of a liquid or solution, used to clean the surface of medical device or the surface of a subject's body, on which microorganisms and/or biofilm-embedded microorganisms live and proliferate. Such cleaning of the medical device or body surface may occur by flushing, rinsing, soaking, or any additional cleaning method known to those skilled in the art, thus removing at least a portion of or reducing the number of microorganisms and/or biofilm-embedded microorganisms attached to at least one surface of the medical device or at least one surface of the subject's body.

Subjects to which administration of the pharmaceutical compositions of the invention is contemplated include, but are not limited to, humans and other primates, mammals including but not limited to non-human mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

### Dosage and Formulation (Pharmaceutical Compositions)

The present invention envisions treating a disease, for example, a bacterial infection, in a subject by the administration of a therapeutic agent, e.g. a humimycin.

Administration of the therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art

The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

The methods of the invention encompass the use of pharmaceutical compositions comprising a humimycin. The pharmaceutical compositions useful for practicing the invention may be administered to deliver a dose of from 1 ng/kg/day and 100 mg/kg/day. In one embodiment, the invention envisions administration of a dose which results in a concentration of the composition of the present invention from 1 μM and 10 μM in a mammal.

Typically, dosages which may be administered in a method of the invention to a mammal, preferably a human, range in amount from 0.5 μg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 1 μg to about 10 mg per kilogram of body weight of the mammal. More preferably, the dosage will vary from about 3 μg to about 1 mg per kilogram of body weight of the mammal.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The compounds and polypeptides (active ingredients) of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the pharmaceutical composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

The present invention also provides pharmaceutical compositions comprising one or more of the compositions described herein. Formulations may be employed in admixtures with conventional excipients, i.e., pharmaceutically acceptable organic or inorganic carrier substances suitable for administration to subject. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

In an embodiment, the composition includes an anti-oxidant and a chelating agent that inhibits the degradation of one or more components of the composition. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the HMW-HA or other composition of the invention in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Discovery of Bioactive Metabolites Encoded by the Human Microbiome Using Primary Sequence Alone

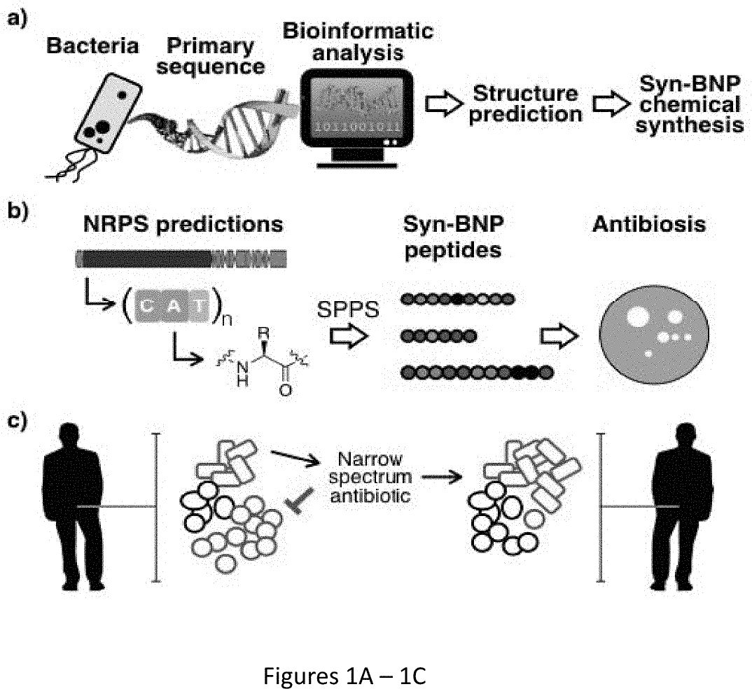

In the current invention, natural product structures are bioinformatically predicted from primary sequence data and produced by chemical synthesis. These bioinformatically inspired compounds are referred to as syn-BNPs for Synthetic Bioinformatic Natural Products (FIG. 1a).

The human microbiome is an exemplary test case for a syn-BNP discovery approach. The human body is home to trillions of bacteria that play important roles in both health and disease. Human cohort microbiome sequencing studies have uncovered numerous correlations between changes in bacterial populations and human pathophysiology (Turnbaugh et al., Nature 2009, 457:480-484; Qin et al., Nature 2014, 513:59-64). Tremendous resources have been allocated to the sequencing and bioinformatic analysis of the human microbiome in an effort to understand the role commensal bacteria play in human physiology (Human Microbiome Project Consortium, Nature 2012, 486:215-221; Chen et al., Database (Oxford) 2010, 2010:baq013). However, the functional characterization of this data, including commensal bacteria-encoded natural product biosynthetic gene clusters, remains very rare.

The materials and methods are now described.

### Materials and Methods

Bioinformatic Prediction of NRPs.

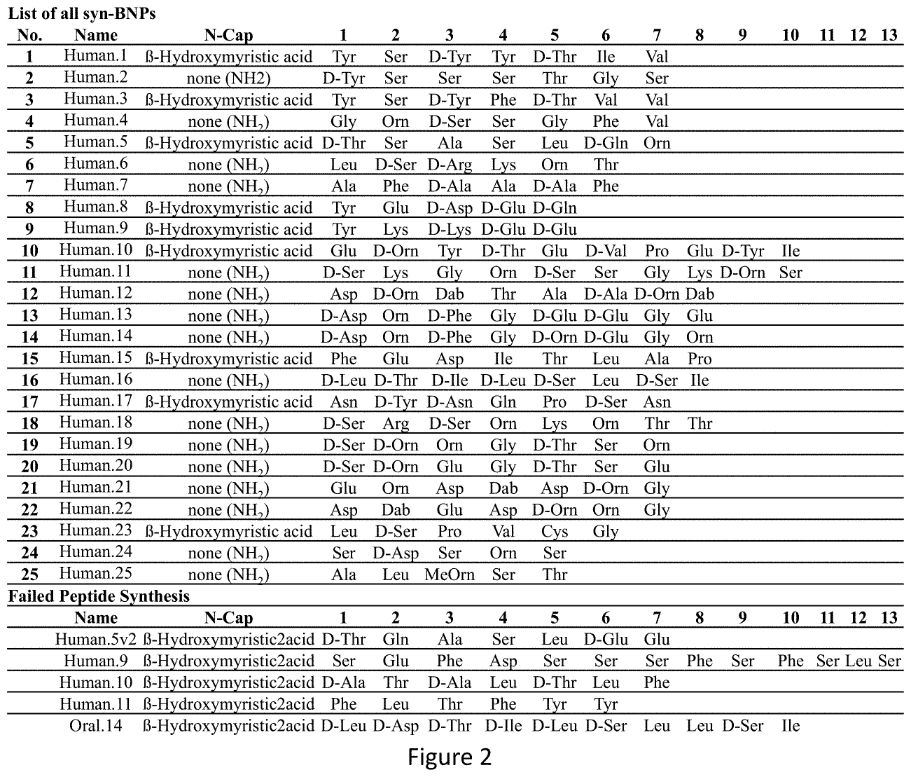

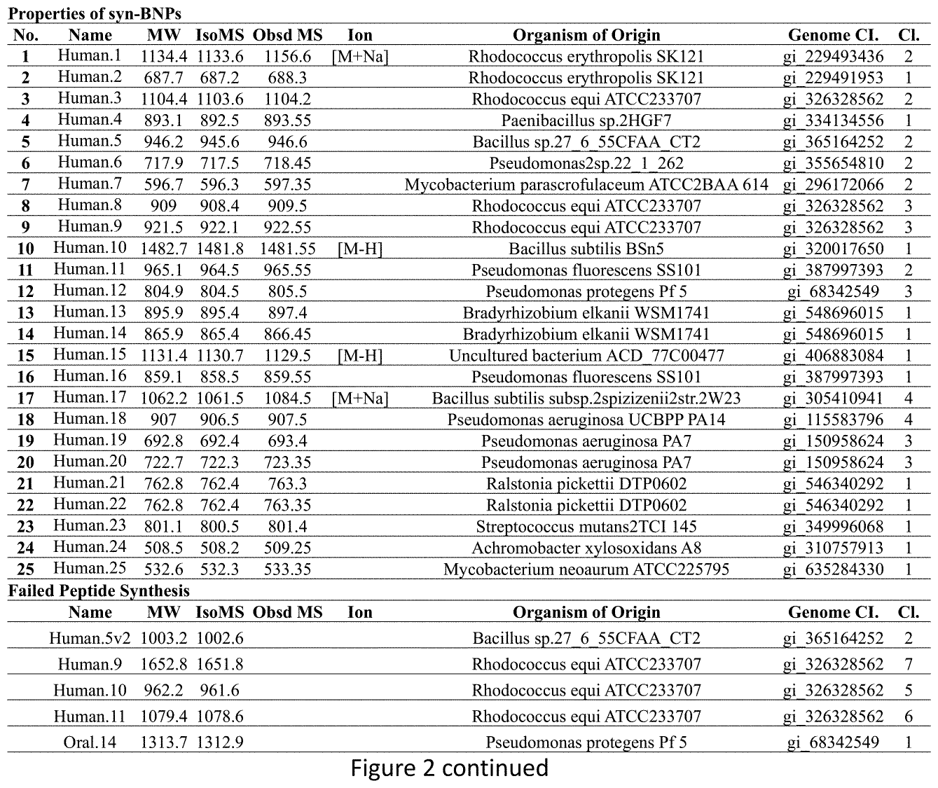

Genome sequences of the human microbiota were downloaded from the NIH Human Microbiome Project (HMP) (Human Microbiome Project Consortium. Nature 2012, 486:215) and the Human Oral Microbiome Database (HOMD) (Chen et al., Database (Oxford) 2010, 2010:baq013). The software package Antibiotics and Secondary Metabolite Analysis Shell (antiSMASH) v2.0 was used for the identification and prediction of NRP biosynthetic gene clusters encoded by these genomes (Blin et al., Nucleic Acids Res. 2013, 41:W204-12). Syn-NRPs originating from the HMP and HOMD databases were named serially as [Human.N] and [Oral.N]. The syn-NRPs discussed herein are listed in FIG. 2. AntiSMASH consults three prediction algorithms to call the amino acid substrate specificity of an adenylation domain (NRPSPredictor2, Stachelhaus code, and Minowa). A consensus prediction refers to the situation wherein two (or all three) algorithms make consistent substrate predictions for a given adenylation domain. In this case the predicted amino acid was used in the synthesis of the syn-BNP. In case of a minor conflict between prediction algorithms we opted for the amino acid with the smaller side-chain, e.g., Val/Leu/Ile and Ser/Thr. In case of major conflicts (e.g., where side-chains were predicted to carry opposite charges), both peptides were synthesized. Tyrosine and phenylalanine prediction made by NRPSPredictor2 or Stachelhaus code were chosen over tryptophan (Trp) predictions made Minowa, as we noticed that Trp is overrepresented in Minowa predictions. Lastly, tyrosine (Tyr) was used at the first residue in place of p-hydroxyphenylglycine (Hpg) in Human.8v1 and v2.

Peptide Synthesis.

Resins for peptide synthesis were purchased from AnaSpec. Coupling reagents (PyBOP) and Nα-Fmoc/side-chain protected amino acids were purchased from P3BioSystems. 3-Hydroxymyristic acids were purchased from TCI America (racemic mixture) and Santa Cruz Biotechnology (pure enantiomers). All other chemical reagents and solvents were purchased from Sigma Aldrich. Reaction vessels were custom made by the Scientific Glassblowing Laboratory at the Department of Chemistry of Yale University.

Pure samples were obtained for 25 of the 30 syn-BNP peptides targeted for chemical synthesis (FIG. 2). Twenty of these peptides were purchased through the custom peptide synthesis service of GenScript Biotech Corporation and five were synthesized in-house. Peptides from GenScript were delivered as lyophilized materials that had been HPLC-purified and MS-verified (MALDI). All pure peptides were dissolved in DMSO at 12.8 mg/mL as stock solutions and stored at −20° C. In-house peptide syntheses, including humimycin A and B, were built on Wang resin (Wang, J. Am. Chem. Soc. 1973, 95:1328) following standard Fmoc/tBu SPPS methods. The first amino acid (6 equiv.) was activated using DIC (3 equiv.) in 10% DMF/DCM (0° C.), added to the resins in the presence of DMAP as a catalyst (0.1 equiv.) and shaken under nitrogen (4 h at 0° C.). Unreacted resins were capped using acetic anhydride in pyridine (1 h). Fmoc removal was accomplished using three rounds of treatment with 20% piperidine in DMF (15, 10, and 5 min. each). All ensuing amino acids were coupled twice. In each coupling an Nα-Fmoc and side-chain protected amino acid was activated using a mixture of PyBOP (4 equiv.) and DIEA (8 equiv.), followed by reaction with the peptide on-resin (1 h). Peptides were cleaved by 95% TFA supplemented with TIS and H2O (2.5% of each, v/v) for 2 h, concentrated to approximately 10% of the original volume, diluted with aqueous MeCN (75%, v/v), passed through a 0.45 μm filter and HPLC-purified. All purified peptides were examined by LC/MS (ESI).

Characterization of the Humimycins.

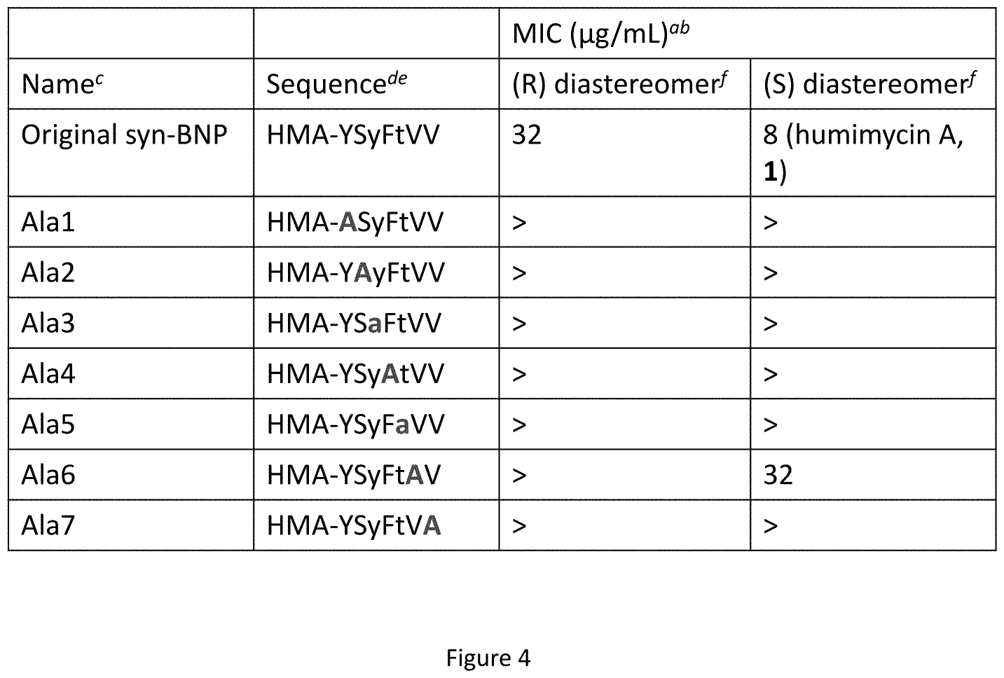

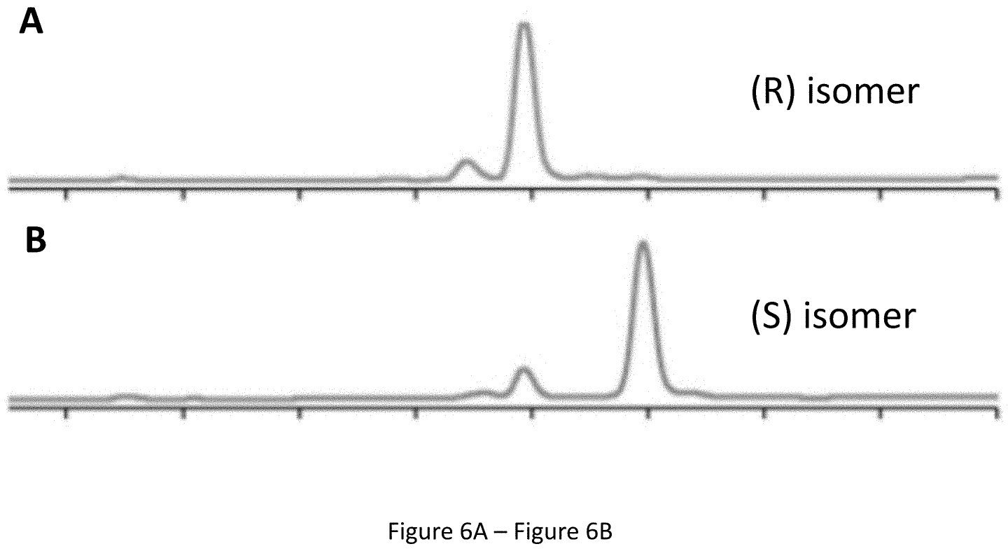

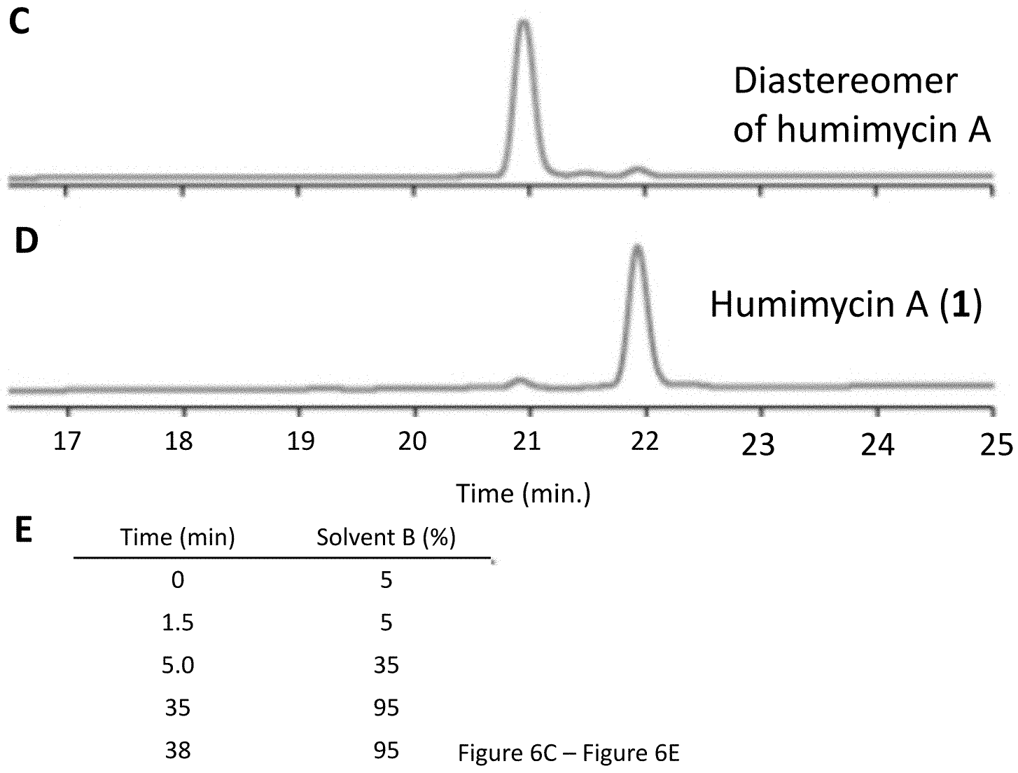

A racemic mixture of 3-hydroxymyristic acid was used for N-terminal modification in our initial syntheses of all syn-BNPs. Humimycin A diastereomers showed different MIC values when tested against MRSA USA300 (FIG. 4). The absolute stereochemistry of the most active diastereomer was determined by comparing HPLC-purified peptides from the bulk synthesis to independent batches of small-scale syntheses using enantiopure (R) and (S)-3-hydroxymyristic acid (FIG. 6). The more potent (S)-isomer is referred to as humimycin A (1). In the case of humimycin B the analogous (S)-isomer was purified and is referred to as compound 2 elsewhere herein. Humimycin A (1) HRMS: m/z calculated for [M+Na]+ (C₅₈H₈₅N₇O₁₄Na): 1126.6052, found: 1126.6021. Humimycin B (2) HRMS: m/z calculated for [M−H]− (C₅₉H₈₆N₇O₁₅): 1132.6182, found: 1132.6194.

Syn-BNP Screening.

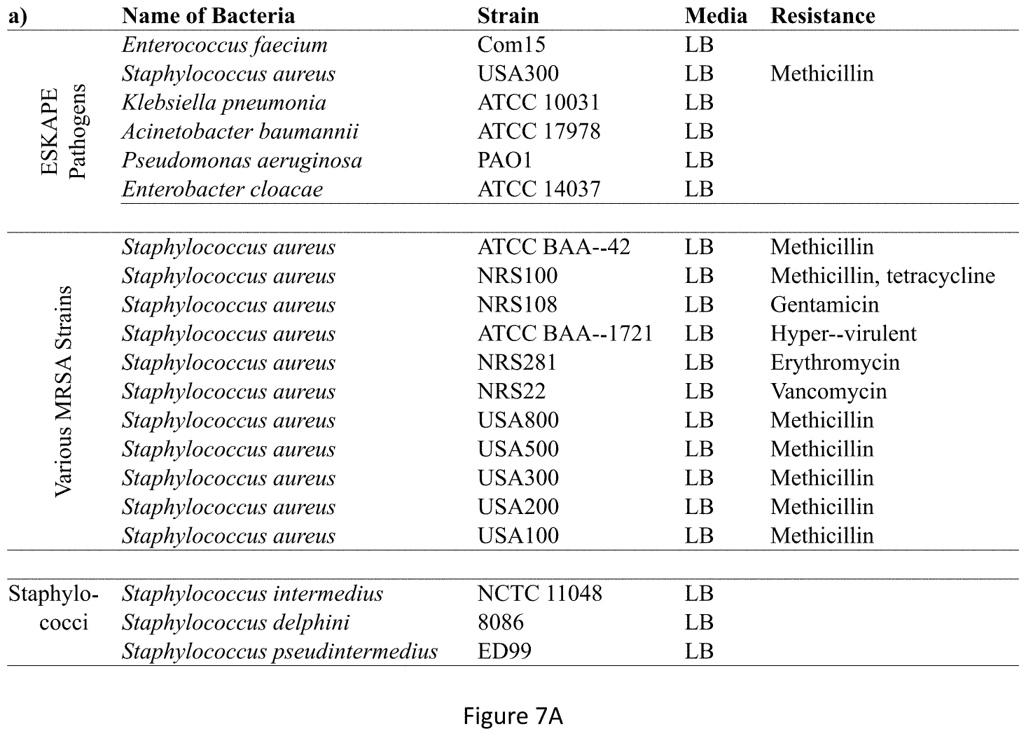

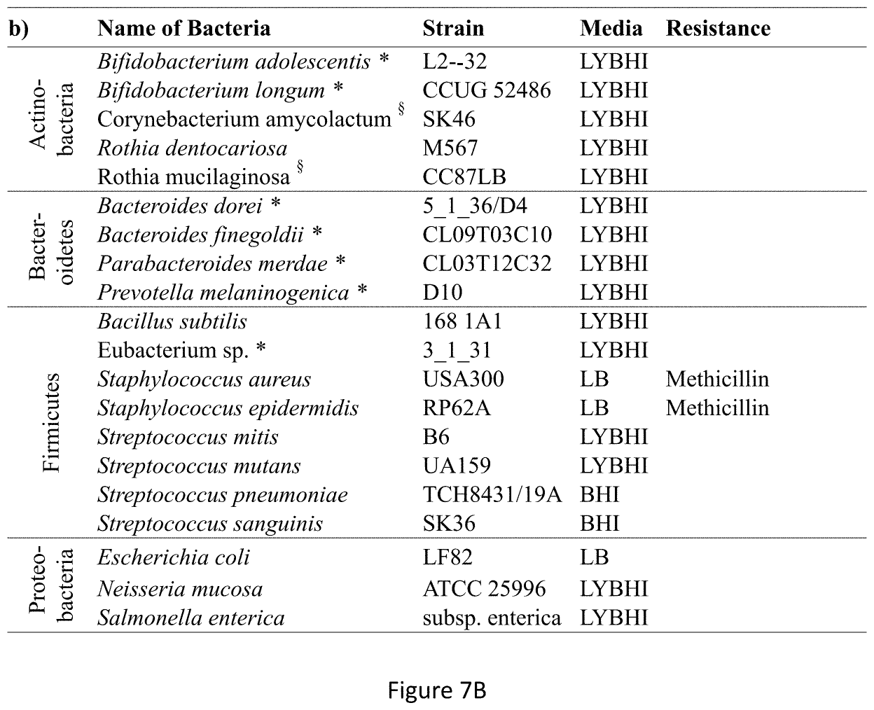

Syn-BNPs were screened against a panel of commensal and pathogenic bacteria covering the four major phyla associated with the human microbiome. This included five Actinobacteria, four Bacteroidetes, six Firmicutes and three Proteobacteria species. All peptides were tested in duplicate for antibiosis activity. Assays were performed in microtiter plates, wherein each well contained growth media (see FIG. 7 for a list of media used) (100 μL), syn-BNP (32 μg/mL) and bacteria diluted 1,000-fold from a stationary phase culture. Binary antibiosis results for most bacteria were determined by visual inspection after static incubation at 37° C. for 18 h. P. melaninogenica and Eubacterium sp. 3_1_31 were grown for 36 h, and C. amycolactum was grown for 60 h. Specific MICs were determined for syn-BNPs that inhibited bacterial growth in this initial screen (see Susceptibility assays, part a). Bacteria species associated with the human flora were obtained from BEI Resources.

Susceptibility Assays.

a) Standard assays. Minimal inhibitory concentration (MIC) assays were performed in duplicate in 96-well microtiter plates based on the protocol recommended by Clinical and Laboratory Standards Institute (CLSI, 2012). DMSO stock solutions of syn-BNPs (12.8 mg/mL) were added to the first well in a row and serially diluted (2 fold per transfer) across the microtiter plate. The last well was reserved for a peptide-free control. Overnight cultures of bacteria were diluted 5,000-fold and 50 μL was used as an inoculum in each well. MIC values were determined by visual inspection after 18 h incubation (37° C., static growth).

b) Synergy assays. Synergistic β-lactam-humimycin activities were assessed through a two-dimensional (2D) susceptibility assay. Two fold serial dilutions were carried out as described above. Carbenicillin (a β-lactam antibiotic) was diluted serially from left to right, and humimycin was diluted serially from top to bottom. The highest concentration tested for both antibiotics was 32 μg/mL. Fractional inhibitory concentration (FIC) is defined as the ratio of the apparent synergistic MIC divided by the MIC of the antibiotic measured alone.

Selection of Humimycin A Resistant Mutants.

A single S. aureus USA300 colony (the parent) from a freshly struck plate was inoculated into LB medium and grown overnight at 37° C. Part of the overnight culture (4 mL) was spun down and kept frozen at −20° C. The rest of the overnight culture was diluted 100-fold, supplemented with humimycin A at 20 μg/mL (2.5×MIC) and 100 μL aliquots was distributed into 200 unique microtiter plate wells. Growth was observed in 50 wells after overnight incubation, indicating the presence of bacteria with mutation(s) conferring humimycin A resistance. Approximately 2 μL of culture from each of these wells was used to inoculate freshly prepared 100 μL aliquots of LB media supplemented with humimycin A (20 μg/mL). The resulting cultures after overnight incubation were struck out for single colonies on LB/agar plates supplemented with humimycin A (20 μg/mL) for single colonies.

Genome Sequencing.

Single colonies of 23 humimycin A resistant mutants as well as the USA300 parent were individually inoculated into 4 mL of LB media free of any antibiotics. After overnight incubation cells were collected by centrifugation. DNA extractions were performed using a MasterPure Purification Kit (EpiCentre Biotechnologies). Multiplex sequencing libraries were prepared from the resulting genomic DNA using a Nextera XT DNA Sample Preparation Kit (FC-131-1024) with Nextera XT Index kit (FC-131-1001) based on protocols provided by the manufacturer (Illumina). Briefly, the genomic DNA was treated with RNase and quantified using the Qubit dsDNA HS Assay System (Q32854, ThermoFisher Scientific). Tagmentation and PCR amplification proceeded according to the manufacturer's protocol, after which the quality and size of the libraries were verified using HS D1000 ScreenTape (TapeStation 2200, Agilent Technologies). Libraries were pooled at equimolar concentrations and column purified by NucleoSpin Gel and PCR Clean-up (MN-750609-250, Macherey-Nagel). The resulting tagged DNA library was size-selected by E-Gel (Life Technologies) and the 450 bp band was excised. The final library pool was checked for molarity on TapeStation and sequenced using MiSeq Reagent Kit v3 (MS-102-3003, Illumina).

Mutation (SNP) Identification.

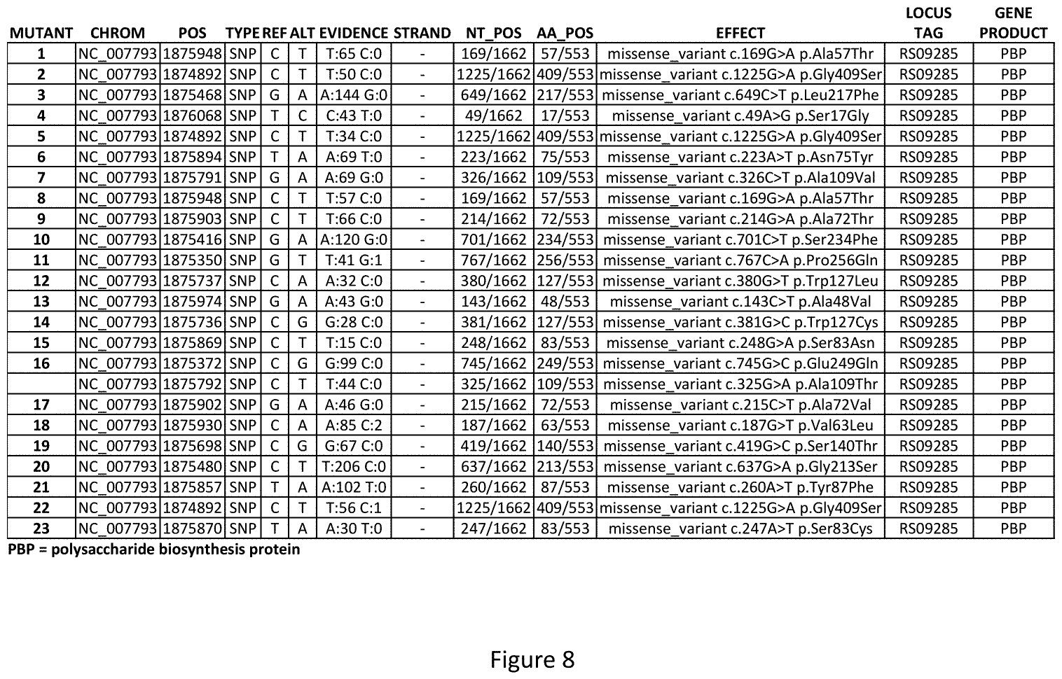

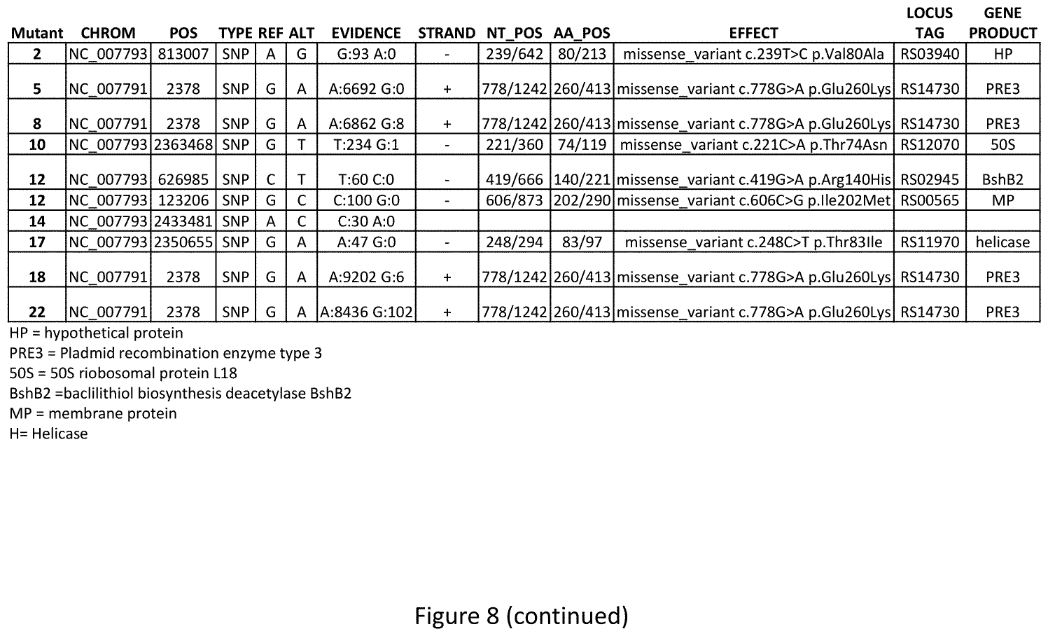

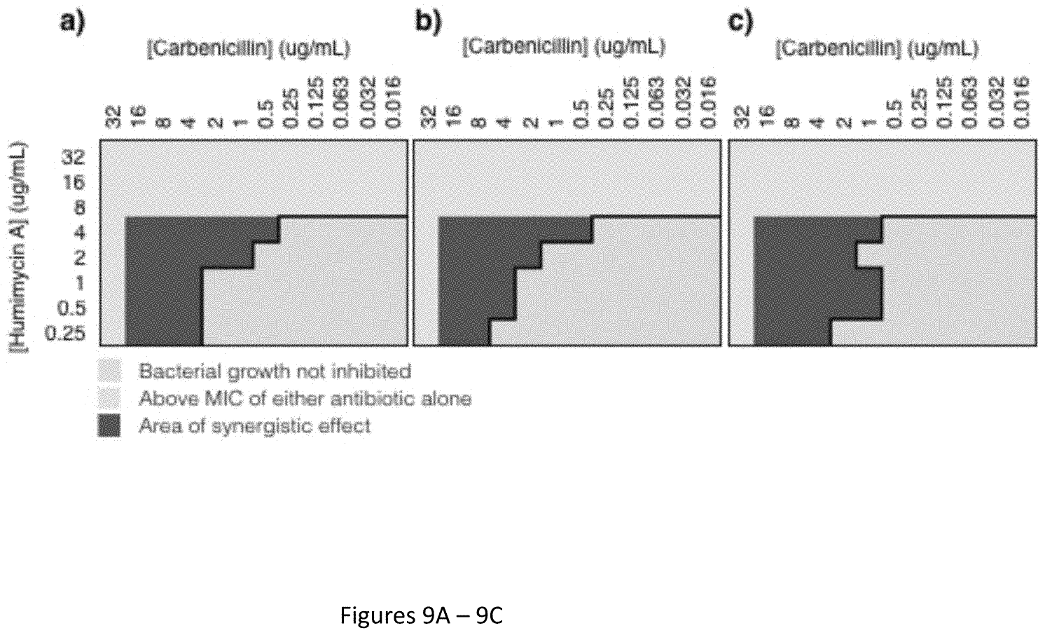

De-barcoded MiSeq reads were assessed for mutations by comparing each read against the reference genome of Staphylococcus aureus USA300 FPR3757 (RefSeq assembly accession: GCF_000013465.1). All reads were mapped to the reference genome using SNIPPY for the identification of variants. SNIPPY is a wrapper of several programs including freebayes (Garrison and Marth, 2012, arXiv:1207.3907). Single-nucleotide polymorphisms (SNP) observed in the parent strain were then subtracted from those observed in the humimycin A resistant strains, resulting in a final list of SNPs (FIG. 8).

The results are now described.

### Natural Product Structures are Bioinformatically Predicted From Primary Sequence Data and Then Produced by Chemical Synthesis (Synthetic-Bioinformatic Natural Products, Syn-BNPs)

Syn-BNPs encoded by the human microbiota stems have potential use as therapeutics and as tools for improving the understanding of human microbiome functions. Antibiotics can serve both as medicines and modulators of the composition of the human microbiome (FIG. 1c). Predicted syn-BNPs from the human microbiome were screened for antibacterial activity against human associated commensal and pathogenic bacteria.

Systematic bioinformatics analyses of sequenced bacterial genomes indicate that nonribosomal peptides (NRPs) are one of the most common and diverse families of complex secondary metabolites produced by bacteria (Cimermancic et al., Cell 2014, 158:412421; Charlop-Powers et al. Elife 2015, 4: e05048; Doroghazi et al., Nat. Chem. Biol. 2014, 10:963-968). NRPs are encoded by modular, often collinear biosynthetic systems, where each module (or unique set of domains) in a megasynthetase incorporates one amino acid into a growing peptide (Finking and Marahiel, Annu. Rev. Microbiol. 2004, 58:453-488). Over the past two decades, a number of structure-based models have been developed for predicting the identity, order, and modification of the amino acids comprising an NRP based solely on the primary sequence of NRP megasynthetases (Stachelhaus et al., Chem. Biol. 1999, 6:493-505; Minowa et al., J. Mol. Biol. 2007, 368:1500-1517; Rottig et al., Nucleic Acids Res. 2011, 39:W362-W367; Weber et al., Nucleic Acids Res. 2015, 43:W237-W243). Concurrently, solid phase peptide synthesis (SPPS) has developed to the point where synthesis of structurally diverse peptides has become rapid and economical, making NRP gene clusters an ideal test case for a syn-BNP approach.

Small molecules encoded by non-ribosomal peptide synthetase (NRPS) gene clusters found in the genomes of human associated microbiota (FIG. 1b) were accessed using this approach. As short NRPs are often highly modified and are therefore not easily accessible using SPPS alone (Walsh et al., Angew. Chem. Int. Ed. 2013, 52:7098-7124), the focus was on large NRPs. Genomic sequence data from human (commensal and pathogenic) associated bacteria (Human Microbiome Project Consortium, Nature 2012, 486:215-221; Chen et al., Database (Oxford) 2010, 2010:baq013) were bioinformatically queried for gene clusters predicted to encode large NRPs (≥5 residues). This analysis led to identification of 57 unique NRPS gene clusters containing five or more modules. From this collection of gene clusters, those that appeared to be incomplete in the existing sequence data or contained more than one PKS module, thioreductase domain, or any heterocyclization domains were removed. The chemical outputs of the remaining 25 gene clusters, were predicted using three published NRPS prediction algorithms (Stachelhaus, Minowa, and NRPSPredictor2) to produce syn-BNP targets (Stachelhaus et al., Chem. Biol. 1999, 6:493-505; Minowa et al., J. Mol. Biol. 2007, 368:1500-1517; Rottig et al., Nucleic Acids Res. 2011, 39:W362-W367; Weber et al., Nucleic Acids Res. 2015, 43:W237-W243). In instances where these predictions disagreed strongly (e.g., side-chains were predicted to carry opposite charges), multiple syn-BNP peptides were designed and synthesized.

Many NRPs are naturally modified on the N-terminus with small collections of fatty acids, resulting in families of NRPs with similar biological activity. In all cases where NRPS gene clusters were bioinformatically predicted to encode an N-terminally acylated peptide, the syn-BNP was designed to be N-acylated with β-hydroxymyristic acid (HMA), a fatty acid commonly observed in NRPs (Rausch et al., BMC Evol. Biol. 2007, 7:78).

In total, 30 syn-BNPs targets were designed based on gene clusters found in human commensal bacterial sequence data. All peptides were synthesized using standard Fmoc chemistry, followed by TFA deprotection and cleavage from the solid support. The mass of each HPLC purified peptide was verified by MS analysis. After two rounds of synthesis, pure samples were obtained for 25 of the 30 targeted syn-BNPs (FIG. 2).

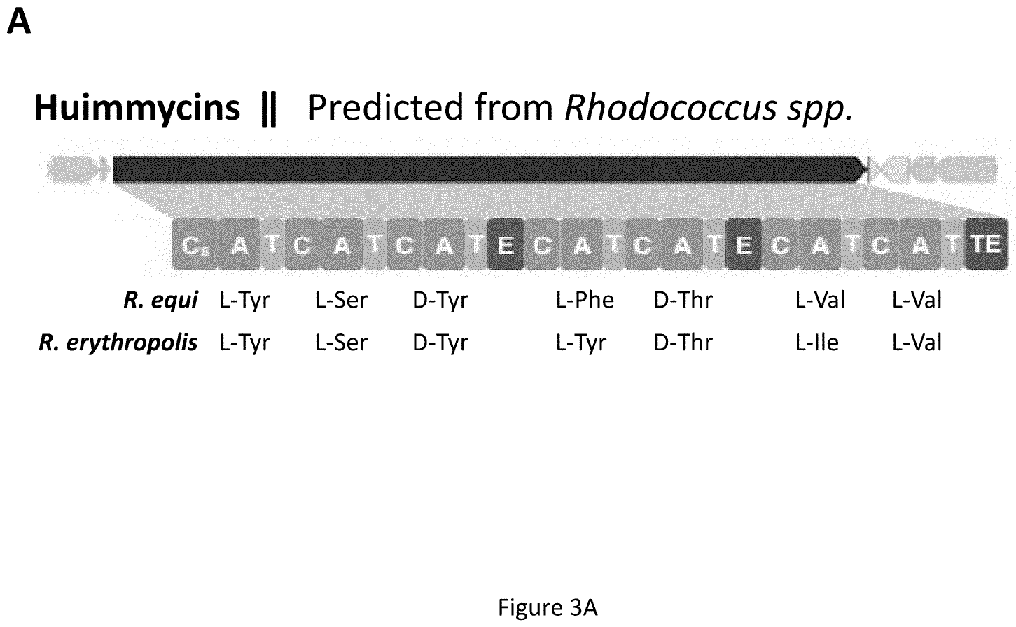

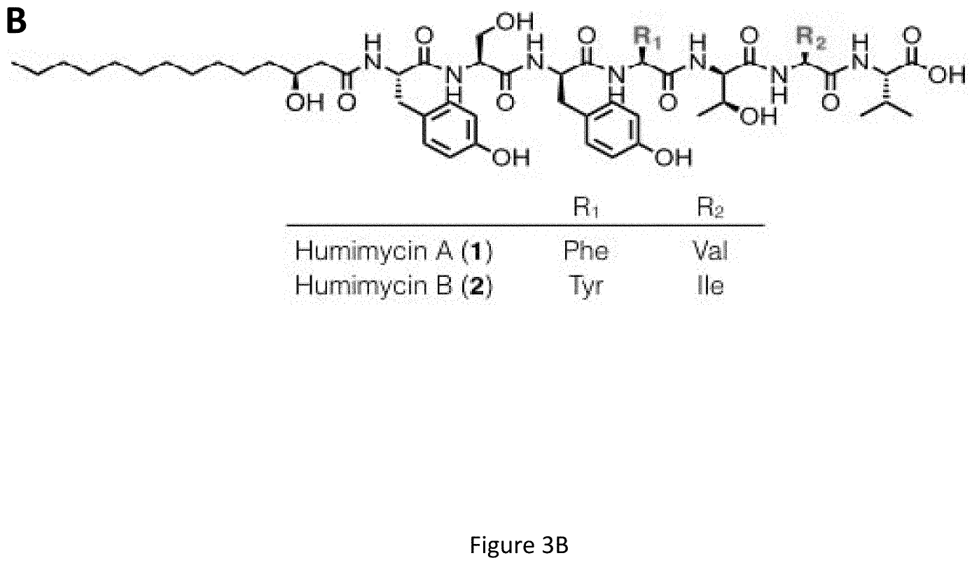

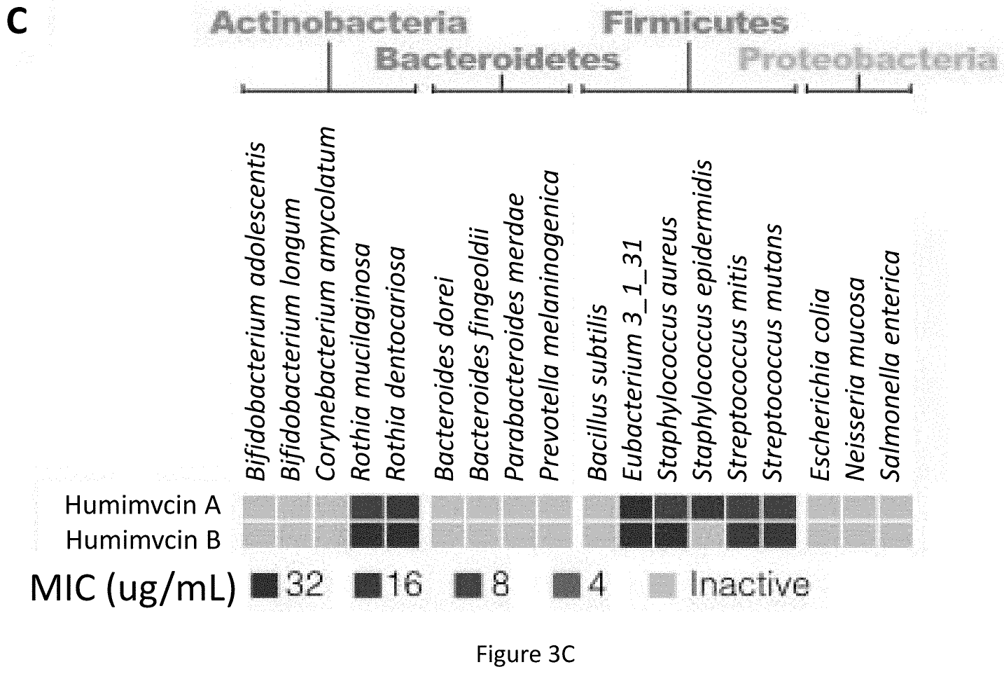

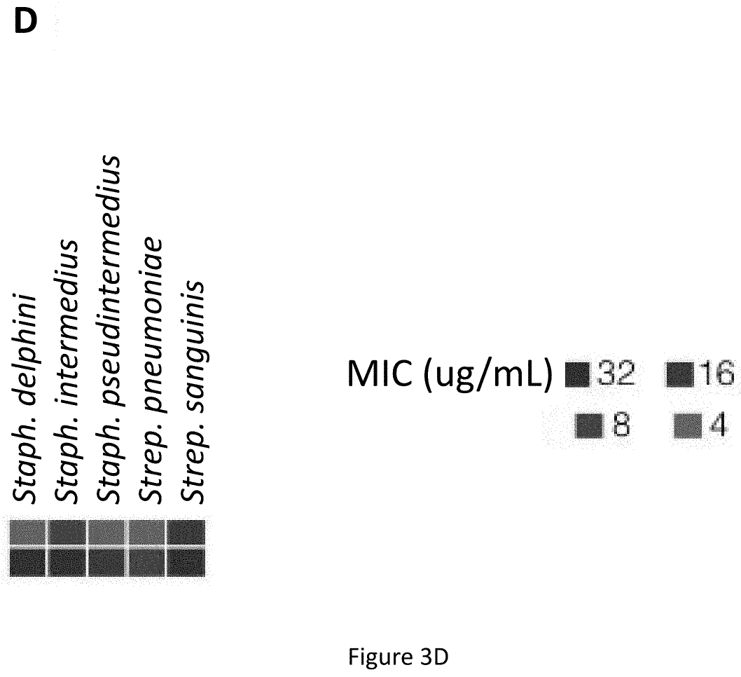

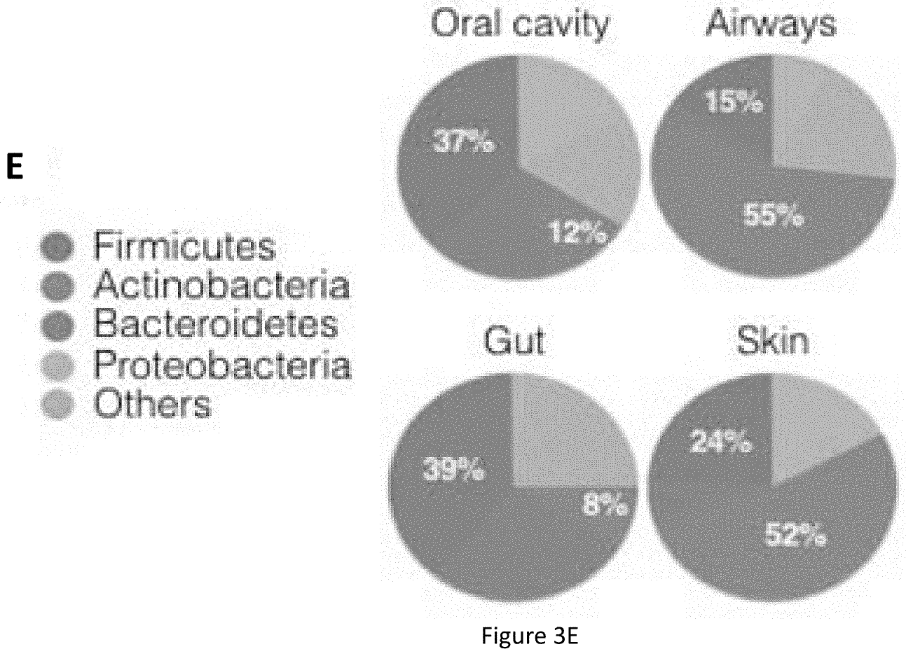

To identify novel antibiotics with potential in vivo roles in shaping the ecology of the human microbiome this collection of syn-BNPs was assayed for antibacterial activity against a panel of common human commensal and pathogenic bacteria. This led to the identification of two antibiotics referred to as humimycin A (1) and B (2) (human microbiome mycin, FIG. 3a). The humimycins were predicted from closely related NRPS gene clusters found in the genomes of Rhodococcus equi and Rhodococcus erythropolis, respectively. Bioinformatic analyses of these two NRPS gene clusters indicated that they encoded hepta-peptides that differed at only the fourth and sixth residues (F/Y and V/I, FIG. 3b). Both syn-BNPs were synthesized with N-terminal HMA modifications, due to the presence of starter condensation domains (Cs), which are associated with acylation of the first amino acid of an NRP (Rausch et al., BMC Evol. Biol. 2007, 7:78).

The humimycins were found to be broadly active against Firmicutes and to show some activity against Actinobacteria (FIG. 3c) when screened for antibiosis against commensal and pathogenic bacteria. This spectrum of activity is interesting in light of the fact that Firmicutes and Actinobacteria dominate the human microbiota of the gut (FIG. 3e) (D'Argenio and Salvatore, Clin Chim Acta 2015, 451:97-102). The humimycins are particularly active against species in the Staphylococcus and Streptococcus genus (FIG. 3d), including common members of the normal human flora such as S. aureus (MIC 8 μg/mL) and S. pneumoniae (MIC 4 μg/mL).

To assess the structure-activity relationship of individual amino acids in humimycins an alanine scan was performed across the peptide portion of humimycin A. No residue could be replaced with alanine without dramatically impacting the potency of the antibiotic (FIG. 4).

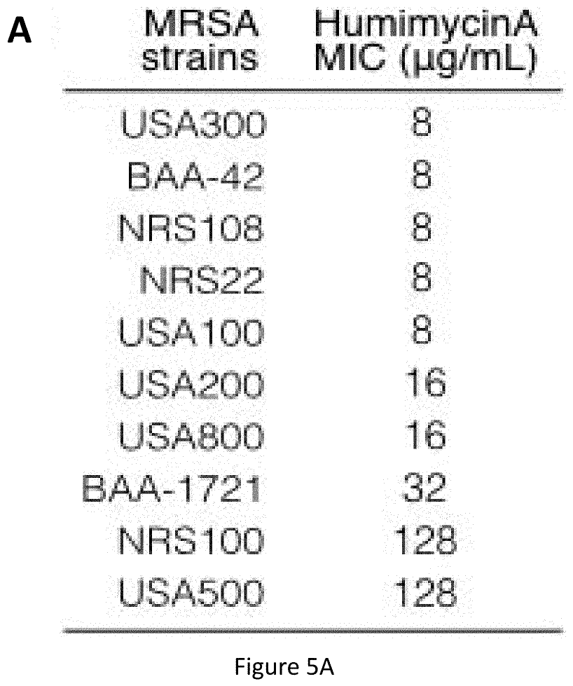

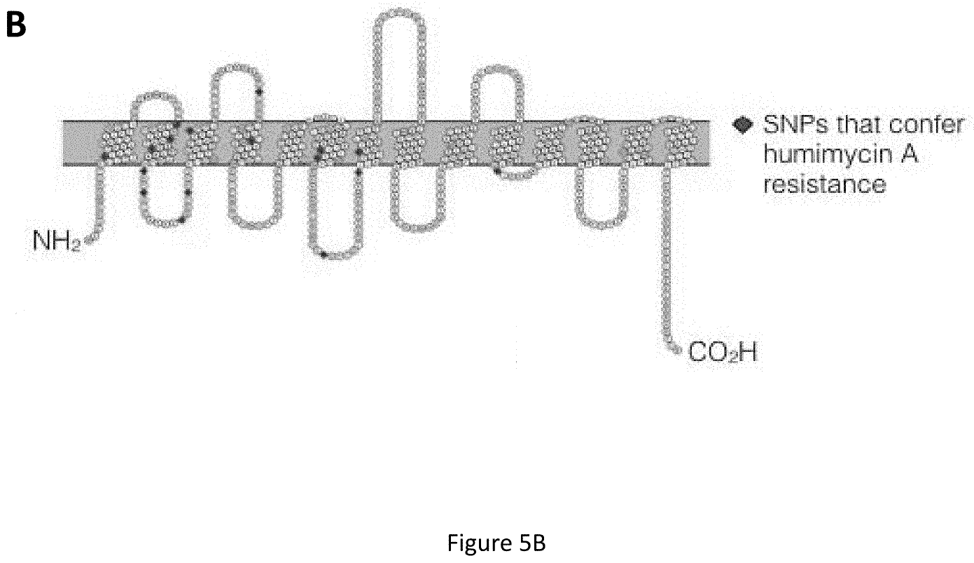

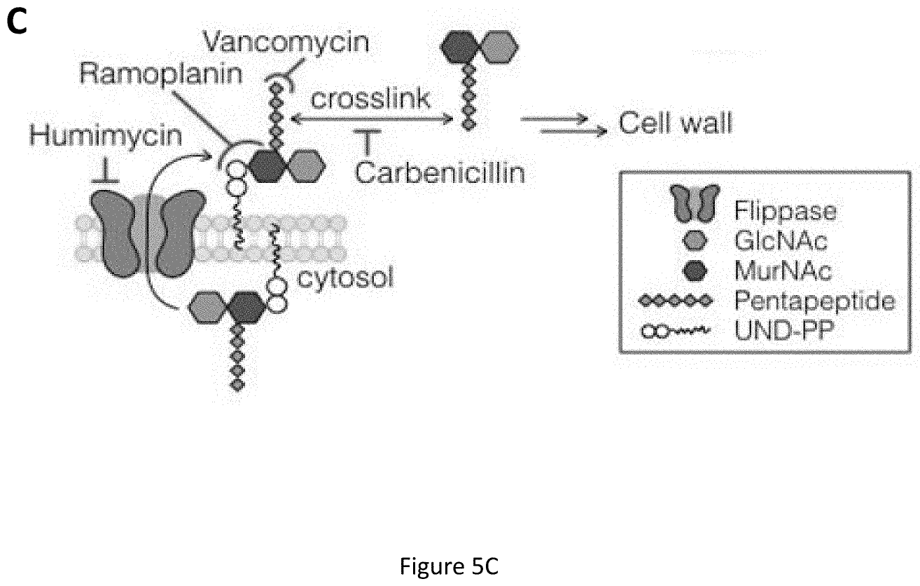

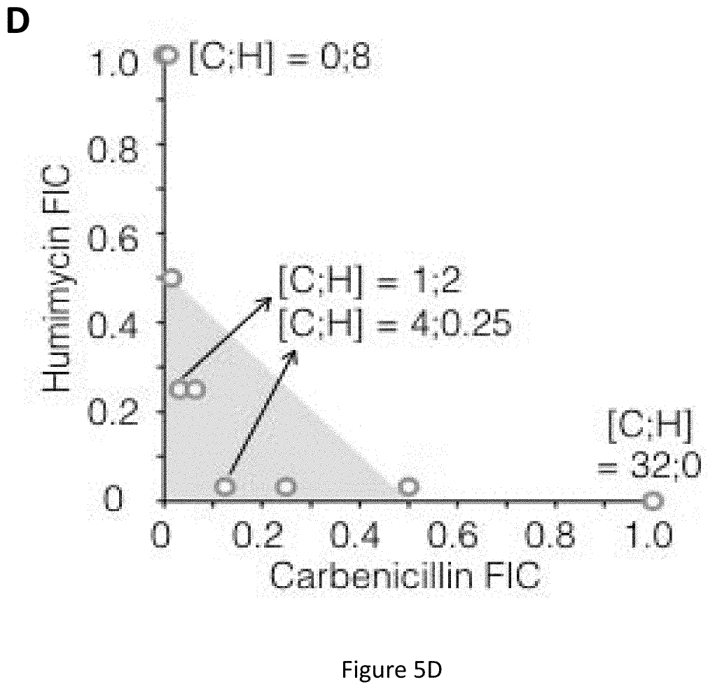

Humimycin A exhibits MICs ranging from 8 to 128 μg/mL against MRSA clinical isolates (FIG. 5a). To study the antibacterial mode of action of the humimycins, S. aureus USA300 mutants were selected that could survive on 2.5 times the MIC (20 μg/mL) and 23 of these resistant mutants were sequenced. Upon comparison of these genome sequences to that of the parent strain, it was found that all 23 resistant mutants contained one non-synonymous mutation in SAV1754, an essential gene in S. aureus (FIG. 5b). Fifteen resistant strains contained no other detectable mutations in their genomes. The gene product of SAV1754 is believed to be a homolog of MurJ, a flippase responsible for the translocation of peptidoglycan precursors from the inside to the outside of the cell (Sham et al., Science 2014, 345:220-222). This is an essential and conserved process in the synthesis of the bacterial cell wall.

While MurJ has been shown to be essential in many bacteria, including many important pathogens, it remains an underexplored antibacterial target (Koyama et al., Molecules 2013, 18:204-224). In a high throughput screen for molecules that could potentiate β-lactam antibiosis against otherwise resistant strains, Merck & Co. identified two synthetic small molecule inhibitors of SAV1754 (DMPI and CDFI) (Huber et al., Chem. Biol. 2009, 16:837-848). The ability of SAV1754 inhibitors to potentiate β-lactam antibiosis is thought to arise from the fact that both antibiotics target the same essential pathway, peptidoglycan biosynthesis (FIG. 5c). Humimycin A exhibits a similar ability to restore β-lactam sensitivity to β-lactam resistant bacteria. For example, the MIC of carbenicillin reduced from 32 to 1 μg/mL in the presence of 2 μg/mL humimycin A (0.25×MIC) against MRSA USA300 (FIG. 5d), the predominant community-associated strain in the US.

The Rhodococcus species that are predicted to encode the humimycins are Gram-positive, obligate aerobic, non-sporulating coccobacilli found throughout the environment. While R. equi has historically been regarded as an opportunistic pathogen seen mostly in animals and immune-compromised patients, the largest study of human microbiota ecology to date reported its presence in the supragingival plaque of 17% (12/70) of the healthy individuals surveyed, albeit in low abundance (0.006%) (Kraal et al., PLoS One 2014, 9:e97279). R. erythropolis has been observed as a part of the normal human nasal, mouth, and eye microbiota (Rasmussen et al. APMIS 2000, 108:663-675; Graham et al. Invest. Ophthalmol. Vis. Sci. 2007, 48:5616-5623). The Rhodococcus species that encode the humimycins are not commonly found in the human gut of healthy individuals. Their occurrence, however, increases dramatically to a median of 30% (up to 66%) in some patients diagnosed with ulcerative colitis (UC) (Lepage et al., Gastroenterology 2011, 141:227-236). The production of an antibiotic with activity against Firmicutes and Actinobacteria could play a role in establishing the overpopulation of R. erythropolis in the UC gut as Firmicutes and Actinobacteria represent nearly half of normal gut microbiota (FIG. 3e) (D'Argenio and Salvatore, Clin Chim Acta 2015, 451:97-102). The discovery of the humimycins provides a testable mechanistic hypothesis for how dysbiosis of gut microbiota might evolve in UC (Jostins et al., Nature 2012, 491:119-124). In general, the role metabolites produced by commensal bacteria play in human physiology is still largely ill defined. The in vitro characterization of commensal bacteria secondary metabolism as outlined here provides a means of developing hypotheses about how commensals bacteria affect human physiology.

The discovery of the humimycins from the human microbiome using the syn-BNP approach highlights the unique state of the field of natural product chemistry today. Extensive biosynthetic studies over the past century have culminated in the ability to begin to predict the structures of many natural products from primary sequence alone. With the development of improved bioinformatic prediction algorithms and the incorporation of more sophisticated chemical and chemo-enzymatic synthesis techniques, the syn-BNP approach will enable broad and rapid access to bioactive metabolites hidden within the ever-growing assemblage of microbial sequence data.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising a peptide, wherein the peptide comprises an amino acid sequence of YSY[X₁]T[X₂]V (SEQ ID NO: 1) or any derivative or analog thereof.

2. The composition of claim 1, wherein the peptide comprises an N-terminal modification.

3. The composition of claim 2, wherein the N-terminal modification comprises an acyl group on the N-terminus of the peptide.

4. The composition of claim 2, wherein the N-terminal modification comprises a fatty acid on the N-terminus of the peptide.

5. The composition of claim 4, wherein the fatty acid is β-hydroxymyristic acid (HMA).

6. The composition of claim 1, wherein X₁ is F or Y.

7. The composition of claim 1, wherein X₂ is V or I.

8. The composition of claim 1, wherein the peptide comprises an amino acid sequence selected from the group consisting of YSYFTVV (SEQ ID NO: 2) and YSYYTIV (SEQ ID NO: 3).

9. The composition of claim 1, wherein Y at position 1, S at position 2, X₁ at position 4, X₂ at position 6, and V at position 7 are in the L configuration.

10. The composition of claim 1, wherein Y at position 3 and T at position 5 are in the D configuration.

11. The composition of claim 1, wherein the composition is an antibiotic.

12. The composition of claim 11 further comprising a pharmaceutical acceptable carrier.

13. The composition of claim 1, wherein the peptide is synthetically synthesized.

14. A method of inhibiting proliferation of a bacterial cell, the method comprising administering to the bacterial cell the composition of claim 1.

15. The method of claim 14, wherein the bacterial cell is Gram− positive.

16. The method of claim 14, wherein the bacterial cell is in a mammal.

17. The method of claim 16, wherein the mammal is a human.

18. The method of claim 16, wherein the mammal is a non-human.
